SEQUENCE LISTING (30 sequences)

SEQ ID NO: 1 - DNA, 1947 bp, Escherichia coli
  CDS (1)..(1947)
atgaatattttaggttttttccagcgactcggtagggcgttacagctccctatcgcggtgctgccggtggcggcactgttgctgcgattcggtcagccagatttacttaacgttgcgtttattgcccaggcgggcggtgcgatttttgataacctcgcattaatcttcgccatcggtgtggcatccagctggtcgaaagacagcgctggtgcggcggcgctggcgggtgcggtaggttactttgtgttaaccaaagcgatggtgaccatcaacccagaaattaacatgggtgtactggcgggtatcattaccggtctggttggtggcgcagcctataaccgttggtccgatattaaactgccggacttcctgagcttcttcggcggcaaacgctttgtgccgattgccaccggattcttctgcctggtgctggcggccatttttggttacgtctggccgccggtacagcacgctatccatgcaggcggcgagtggatcgtttctgcgggcgcgctgggttccggtatctttggtttcatcaaccgtctgctgatcccaaccggtctgcatcaggtactgaacaccatcgcctggttccagattggtgaattcaccaacgcggcgggtacggttttccacggtgacattaaccgcttctatgccggtgacggcaccgcggggatgttcatgtccggcttcttcccgatcatgatgttcggtctgccgggtgcggcgctggcgatgtacttcgcagcaccgaaagagcgtcgtccgatggttggcggtatgctgctttctgttgctgttactgcgttcctgaccggtgtgactgagccgctggaattcctgttcatgttccttgctccgctgctgtacctcctgcacgcactgctgaccggtatcagcctgtttgtggcaacgctgctgggtatccacgcgggcttctctttctctgcgggggctatcgactacgcgttgatgtataacctgccggccgccagccagaacgtctggatgctgctggtgatgggcgttatcttcttcgctatctacttcgtggtgttcagtttggttatccgcatgttcaacctgaaaacgccgggtcgtgaagataaagaagacgagatcgttactgaagaagccaacagcaacactgaagaaggtctgactcaactggcaaccaactatattgctgcggttggcggcactgacaacctgaaagcgattgacgcctgtatcacccgtctgcgccttacagtggctgactctgcccgcgttaacgatacgatgtgtaaacgtctgggtgcttctggggtagtgaaactgaacaaacagactattcaggtgattgttggcgcgaaagcagaatccatcggcgatgcgatgaagaaagtcgttgcccgtggtccggtagccgctgcgtcagctgaagcaactccggcaactgccgcgcctgtagcaaaaccgcaggctgtaccaaacgcggtatctatcgcggagctggtatcgccgattaccggtgatgtcgtggcactggatcaggttcctgacgaagcattcgccagcaaagcggtgggtgacggtgtggcggtgaaaccgacagataaaatcgtcgtatcaccagccgcagggacaatcgtgaaaatcttcaacaccaaccacgcgttctgcctggaaaccgaaaaaggcgcggagatcgtcgtccatatgggtatcgacaccgtagcgctggaaggtaaaggctttaaacgtctggtggaagagggtgcgcaggtaagcgcagggcaaccgattctggaaatggatctggattacctgaacgctaacgcccgctcgatgattagcccggtggtttgcagcaatatcgacgatttcagtggcttgatcattaaagctcagggccatattgtggcgggtcaaacaccgctgtatgaaatcaaaaagtaa

SEQ ID NO: 2 - protein, 648 aa, Escherichia coli
MNILGFFQRLGRALQLPIAVLPVAALLLRFGQPDLLNVAFIAQAGGAIFDNLALIFAIGVASSWSKDSAGAAALAGAVGYFVLTKAMVTINPEINMGVLAGIITGLVGGAAYNRWSDIKLPDFLSFFGGKRFVPIATGFFCLVLAAIFGYVWPPVQHAIHAGGEWIVSAGALGSGIFGFINRLLIPTGLHQVLNTIAWFQIGEFTNAAGTVFHGDINRFYAGDGTAGMFMSGFFPIMMFGLPGAALAMYFAAPKERRPMVGGMLLSVAVTAFLTGVTEPLEFLFMFLAPLLYLLHALLTGISLFVATLLGIHAGFSFSAGAIDYALMYNLPAASQNVWMLLVMGVIFFAIYFVVFSLVIRMFNLKTPGREDKEDEIVTEEANSNTEEGLTQLATNYIAAVGGTDNLKAIDACITRLRLTVADSARVNDTMCKRLGASGVVKLNKQTIQVIVGAKAESIGDAMKKVVARGPVAAASAEATPATAAPVAKPQAVPNAVSIAELVSPITGDVVALDQVPDEAFASKAVGDGVAVKPTDKIVVSPAAGTIVKIFNTNHAFCLETEKGAEIVVHMGIDTVALEGKGFKRLVEEGAQVSAGQPILEMDLDYLNANARSMISPVVCSNIDDFSGLIIKAQGHIVAGQTPLYEIKK

SEQ ID NO: 3 - DNA, 70 bp, Artificial
  primer
gatgaaagcgttatccaaactgaaagcggaagaggccgacgcactttgcgccgaataaatacctgtgacg

SEQ ID NO: 4 - DNA, 69 bp, Artificial
  primer
ttaatcccagctcagaataactttcccggactttacgccccgccctgccactcatcgcagtactgttgt

SEQ ID NO: 5 - DNA, 18 bp, Artificial
  primer
cggtcatgcttggtgatg

SEQ ID NO: 6 - DNA, 21 bp, Artificial
  primer
ttaatcccagctcagaataac

SEQ ID NO: 7 - DNA, 30 bp, Artificial
  primer
ccaagatctggagcttatcgactgcacggt

SEQ ID NO: 8 - DNA, 71 bp, Artificial
  primer
acgccctaccgagtcgctggaaaaaacctaaaatattcatggtctgtttcctgtgtgaaattttatccgct

SEQ ID NO: 9 - DNA, 69 bp, Artificial
  primer
tcacacactctgtagcagatgatctaacaatctgattacagtcagaaaaactcatcgagcatcaaatga

SEQ ID NO: 10 - DNA, 30 bp, Artificial
  primer
ttcagatctgttgtgtctcaaaatctccga

SEQ ID NO: 11 - DNA, 25 bp, Artificial
  primer
ctgacctggcctgctttatgcattt

SEQ ID NO: 12 - DNA, 25 bp, Artificial
  primer
aatctggctgacgcaatcgcagcaa

SEQ ID NO: 13 - DNA, 69 bp, Artificial
  primer
tccacgagatgcggcccaatttacgtcttaggagaagatcaactaagcacttgtctcctgtttactccc

SEQ ID NO: 14 - DNA, 69 bp, Artificial
  primer
aatggcgccgatgggcgccatttttcactgcggcaagaattgctgcttttaagacccactttcacattt

SEQ ID NO: 15 - DNA, 24 bp, Artificial
  primer
agcaggctcttgctcaaccgacaa

SEQ ID NO: 16 - DNA, 24 bp, Artificial
  primer
ggaagtgatgcgctacacccagca

SEQ ID NO: 17 - DNA, 33 bp, Artificial
  primer
cacaaagcttggttctcgtaggaggaataagat

SEQ ID NO: 18 - DNA, 32 bp, Artificial
  primer
tgtgtctagacctcactcatcgtggattcctc

SEQ ID NO: 19 - DNA, 180 bp, Artificial
  hybrid promoter
ctagatctctcacctaccaaacaatgcccccctgcaaaaaataaattcataaaaaacatacagataaccatctgcggtgataaattatctctggcggtgttgacaattaatcatcggctcgtataatgtgtggaattgtgagcgggttctcgtagggggaataagatgaatattttaggt

SEQ ID NO: 20 - DNA, 32 bp, Artificial
  primer
gtaagatctctcatgtttgacagcttatcatc

SEQ ID NO: 21 - DNA, 52 bp, Artificial
  primer
cctaaaatattcatcttattccccctacgagaacccctaagctttctagacg

SEQ ID NO: 22 - DNA, 52 bp, Artificial
  primer
acactctgtagcagatgatctaacaatctgattacaattacgccccgccctg

SEQ ID NO: 23 - DNA, 24 bp, Artificial
  primer
ctgacctggcctgctttatgcatt

SEQ ID NO: 24 - DNA, 26 bp, Artificial
  primer
aatctggctgaccgaatcgcagcaac

SEQ ID NO: 25 - protein, 650 aa, Salmonella typhimurium
MNILGFFQRLGRALQLPIAVLPVAALLLRFGQPDLLNMPFIAQAGGSIFDNLALVFAIGVASSWSKDSAGAAALAGAVGYFVMTKAMVTINPEINMGVLAGIITGLVGGAVYNRWSGIKLPDFLSFFGGKRFVPIATGFFCLVLAAIFGYVWPPVQHGIHAGGEWIVSAGALGSGIFGFINRLLIPTGLHQVLNTIAWFQIGEFTNAAGTVFHGDINRFYAGDGTAGMFMSGFFPIMMFGLPGAALAMYFAAPKERRPMVGGMLLSVAITAFLTGVTEPLEFLFMFLAPLLYLLHAILTGISLFVATLLGIHAGFSFSAGAIDYVLMYNLPAASNNVWMLLVMGVVFFIIYFLLFSAVIRMFNLKTPGREDKVDEMVTEEANSNTEEGLTQLATSYIAAVGGTDNLKAIDACITRLRLTVNDSARVNDAACKRLGASGVVKLNKQTIQVIVGAKAESIGDEMKKVVARGPVAAASADAAHVATPAPAAKPQAVPNAVTIAELVSPITGEVVALDQVPDEAFASKAVGDGVAVKPTDKTVVSPAAGTIVKIFNTNHAFCLETEKGAEIVVHMGIDTVALNGQGFKRLVEEGAEVTAGQPVLELDLDFLNANARSMISPVVCSNSDDFSALVIKADGHVVAGKTPLYEIKSK

SEQ ID NO: 26 - protein, 650 aa, Salmonella typhi
MNILGFFQRLGRALQLPIVVLPVAALLLRFGQPDLLNMPFIAQAGGSIFDNLALVFAIGVASSWSKDSAGAAALAGAVGYFVMTKAMVTINPEINMGVLAGIITGLVGGAVYNRWSGIKLPDFLSFFGGKRFVPIATGFFCLVLAAIFGYVWPPVQHGIHAGGEWIVSAGALGSGIFGFINRLLIPTGLHQVLNTIAWFQIGEFTNAAGTVFHGDINRFYAGDGTAGMFMSGFFPIMMFGLPGAALAMYFAAPKERRPMVGGMLLSVAITAFLTGVTEPLEFLFMFLAPLLYLLHAILTGISLFVATLLGIHAGFSFSAGAIDYVLMYNLPAASNNVWMLLVMGVVFFIIYFLLFSAVIRMFNLKTPGREDKVDEMVTEEANSNTEEGLTQLATSYIAAVGGTDNLKAVDACITRLRLTVNDSARVNDAACKRLGASGVVKLNKQTIQVIVGAKAESIGDEMKKVVARGPVAAASADAAHVATPAPAAKPQAVPNAVTIAELVSPITGEVVALDQVPDEAFASKAVGDGVAVKPTDKTVVSPAAGTIVKIFNTNHAFCLETEKGAEIVVHMGIDTVALNGQGFKRLVEEGAEVTAGQPVLELDLDFLNANARSKISPVVCSNSDDFSALVIKADGHVVAGQTPLYEIKSK

SEQ ID NO: 27 - protein, 648 aa, Shigella flexneri
MNILGFFQRLGRALQLPIAVLPVAALLLRFGQPDLLNVAFIAQAGGAIFDNLALIFAIGVASSWSKDSAGAAALAGAVGYFVLTKAMVTINPEINMGVLAGIITGLVGGAAYNRWSDIKLPDFLSFFGGKRFVPIATGFFCLVLAAIFGYVWPPVQHAIHAGGEWIVSAGALGSGIFGFINRLLIPTGLHQVLNTIAWFQIGEFTNAAGTVFHGDINRFYAGDGTAGMFMSGFFPIMMFGLPGAALAMYFAAPKERRPMVGGMLLSVAVTAFLTGVTEPLEFLFMFLAPLLYLLHALLTGISLFVATLLGIHAGFSFSAGAIDYALMYNLPAASQNVWMLLVMGVIFFAIYFVVFSLVIRMFNLKTPGREDKEDEIVTEEANSNTDSCLTQLATNYIAAVGGTDNLKAIDACITRLRLTVADSARVNDTMCKRLGASGVVKLNKQTIQVIVGAKAESIGDAMKKVVARGPVAAASAEATPATAAPVAKPQAVPNAVSIAELVSPITGDVVALDQVPDEAFASKAVGDGVAVKPTDKIVVSPAAGTIVKIFNTNHAFCLETEKGAEIVVHMGIDTVALEGKGFKRLVEEGAQVSAGQPILEMDLDYLNANARSMISPVVCSNIDDFSGLLIKAQGHVVAGQTPLYEIKK

SEQ ID NO: 28 - protein, 651 aa, Klebsiella pneumoniae
MNILGFFQRLGRALQLPIAVLPVAALLLRFGQPDLLNVPFIAQAGGAIFDNLALIFAIGVASSWSKDNAGSAALAGAVGYFVMTKAMVTINPEINMGVLAGIITGLVAGAVYNRWAGIKLPDFLSFFGGKRFVPIATGFFCLILAAIFGYVWPPVQHAIHSGGEWIVSAGALGSGIFGFINRLLIPTGLHQVLNTIAWFQIGEFTNAAGTVFHGDINRFYAGDGTAGMFMSGFFPIMMFGLPGAALAMYLAAPKARRPMVGGMLLSVAITAFLTGVTEPLEFLFLFLAPLLYLLHAVLTGISLFIATALGIHAGFSFSAGAIDYVLMYSLPAASKNVWMLLVMGVVFFFVYFLLFSAVIRMFNLKTPGREDKAADVVTEEANSNTEEGLTQLATSYIAAVGGTDNLKAIDACITRLRLTVGDSAKVNDAACKRLGASGVVKLNKQTIQVIVGAKAESIGDEMKKVVTRGPVAAAAAAPAGNVATAAPAAKPQAVANAKTVESLVSPITGDVVALEQVPDEAFASKAVGDGIAVKPTDNIVVAPAAGTVVKIFNTNHAFCLETNNGAEIVVHMGIDTVALEGKGFKRLVEEGTDVKAGEPILEMDLDFLNANARSMISPVVCSNSDDYSALVILASGKVVAGQTPLYEIKGK

SEQ ID NO: 29 - protein, 677 aa, Yersinis pestis
MSILGYLQKVGRALMVPVATLPAAAILMGVGYWIDPVGWGADNALAALFIKSGAAIIENMSVLFAIGVAYGMSKDKDGAAALTGFVGFLVLTTLCSPAAVSMIKQIPLDQVPAAFGKIENQFVGILVGIISAELYNRFSGVELPKALSFFSGRRLVPILTSFLMIAVAFMLMYIWPLIYNALVTFGEYIKDLGSVGAGIYAFFNRLLIPVGLHHALNSVFWFDVAGINDIPNFLGGQESINKGTGIVGITGRYQAGFFPIMMFGLPGAALAIYHCARPENKAKVAGIMMAGAFAAFFTGITEPLEFSFMFVAPVLYFLHAVLTGISVFIAASMHWIAGFGFSAGLVDMVLSSRNPLATQWYMLIPQGLIFFVIYYLVFRFTIQKFNLLTPGRELAVEGSEEDGYDVNVDKTPAVNESEINSLARRYIGAIGGSDNLTAIDACITRLRLNVKDSALVNDSVAKRLGASGVIRLNKQSVQIIVGTRAELIAAAMRTVLAGGPIPAASSNAAPTGARPQAVINTAKTASLVLVSPITGDVVPLAQVPDEAFASKAVGEGVAIRPTDKIVVSPASGTIVKIFNTDHAFCLETETGAEIVVHIGIDTVKLNGQGFTRLVEEGTTVVAGQPVLELDLAYLNANAHSMISPVVVSNIDDYAGISLLASGSVVAGQSQLFEIRGK

SEQ ID NO: 30 - protein, 677 aa, Yersinis pseudotuberculosis
MSILGYLQKVGRALMVPVATLPAAAILMGVGYWIDPVGWGADNALAALFIKSGAAIIENMSVLFAIGVAYGMSKDKDGAAALTGFVGFLVLTTLCSPAAVSMIKQIPLDQVPAAFGKIENQFVGILVGIISAELYNRFSGVELPKALSFFSGRRLVPILTSFLMIAVAFMLMYIWPLIYNALVTFGEYIKDLGSVGAGIYAFFNRLLIPVGLHHALNSVFWFDVAGINDIPNFLGGQESINKGTGIVGITGRYQAGFFPIMMFGLPGAALAIYHCARPENKAKVAGIMMAGAFAAFFTGITEPLEFSFMFVAPVLYFLHAVLTGISVFIAASMHWIAGFGFSAGLVDMVLSSRNPLATQWYMLIPQGLIFFVIYYLVFRFTIQKFNLLTPGRELAVEGSEEDGYDVNVDKTPAVNESEINGLARRYIGAIGGSDNLTAIDACITRLRLNVKDSALVNDSVAKRLGASGVIRLNKQSVQIIVGTRAELIAAAMRTVLAGGPIPAASSNAAPTGARPQAVINTAKTASLVLVSPITGDVVPLAQVPDEAFASKAVGEGVAIRPTDKIVVSPASGTIVKIFNTDHAFCLETETGAEIVVHIGIDTVKLNGQGFTRLVEEGTTVVAGQPVLELDLAYLNANAHSMISPVVVSNIDDYAGISLLASGSVVAGQSQLFEIRGK

(19) United States
(12) Patent Application Publication
(10) Pub. No.: US 2007/0212764 A1
(43) Pub. Date: Sep. 13, 2007

(54) METHOD FOR PRODUCING L-AMINO ACIDS USING BACTERIUM OF THE ENTEROBACTERIACEAE FAMILY

(76) Inventors: Leonid Romanovich Ptitsyn, Moscow (RU); Irina Borisovna Altman, Moscow (RU); Veronika Aleksandrovna Kotliarova, Moscow (RU); Yury Ivanovich Kozlov, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/275,507

(22) Filed: Jan. 11, 2006

Related U.S. Application Data
(60) Provisional application No. 60/703,414, filed on Jul. 29, 2005.

(30) Foreign Application Priority Data
Jan. 19, 2005 (RU) 2005101111

(51) Int. Cl. C12P 13/18; C12P 13/04; C12P 13/24; C12P 13/22; C12P 13/20; C12P 13/14; C12P 13/16; C12P 13/12; C12P 13/10; C12N 1/21

(52) U.S. Cl. 435/106; 435/107; 435/108; 435/109; 435/110; 435/111; 435/112; 435/113; 435/114; 435/252.3; 435/252.33

(57) ABSTRACT

There is disclosed a method for producing an L-amino acid, for example L-threonine, L-lysine, L-histidine, L-phenylalanine, L-arginine, L-tryptophan, or L-glutamic acid, using a bacterium of the Enterobacteriaceae family, wherein the bacterium has been modified to enhance an activity of N-acetylglucosamine permease encoded by the nagE gene.

This application claims priority under 35 U.S.C. § 119(a) to Russian patent application 2005101111, filed on Jan. 19, 2005, and under 35 U.S.C. §119(e) to U.S. provisional application 60/703,414, filed on Jul. 29, 2005, the entireties of both are hereby incorporated by reference. The Sequence Listing filed herewith on compact disk is also incorporated by reference (File Name: US-196 Seq List; File Size: 52 KB; Date Created: Jan. 11, 2006)

### BACKGROUND OF THE INVENTION

1. Technical Field

The present invention relates to a method for producing an L-amino acid by fermentation, and more specifically to genes which aid in this fermentation. These genes are useful for the improving L-amino acid production, for example, for production of L-threonine, L-lysine, L-histidine, L-phenylalanine, L-arginine, L-tryptophan, and L-glutamic acid.

2. Background Art

Conventionally, L-amino acids are industrially produced by fermentation methods utilizing strains of microorganisms obtained from natural sources, or mutants thereof. Typically, the microorganisms are modified to enhance production yields of L-amino acids.

Many techniques to enhance L-amino acid production yields have been reported, including transformation of microorganisms with recombinant DNA (see, for example, U.S. Pat. No. 4,278,765). Other techniques for enhancing production yields include increasing the activities of enzymes involved in amino acid biosynthesis and/or desensitizing the target enzymes of the feedback inhibition by the resulting L-amino acid (see, for example, WO 95/16042 or U.S. Pat. Nos. 4,346,170, 5,661,012 and 6,040,160).

Strains useful in production of L-threonine by fermentation are known, including strains with increased activities of enzymes involved in L-threonine biosynthesis (U.S. Pat. Nos. 5,175,107; 5,661,012; 5,705,371; 5,939,307; EP 0219027), strains resistant to chemicals such as L-threonine and its analogs (WO 01/14525A1, EP 301572 A2, U.S. Pat. No. 5,376,538), strains with target enzymes desensitized to feedback inhibition by the produced L-amino acid or its by-products (U.S. Pat. Nos. 5,175,107; 5,661,012), and strains with inactivated threonine degradation enzymes (U.S. Pat. Nos. 5,939,307; 6,297,031).

The known threonine-producing strain Escherichia coli VKPM B-3996 (U.S. Pat. Nos. 5,175,107 and 5,705,371) is presently one of the best known threonine producers. To construct the VKPM B-3996 strain, several mutations and a plasmid, described below, were introduced into the parent strain E. coli K-12 (VKPM B-7). A mutant thrA gene (mutation thrA442) encodes aspartokinase homoserine dehydrogenase I, which is resistant to feedback inhibition by threonine. A mutant ilvA gene (mutation ilvA442) encodes threonine deaminase which has a decreased activity, and results in a decreased rate of isoleucine biosynthesis and a leaky phenotype of isoleucine starvation. In bacteria containing the ilvA442 mutation, transcription of the thrABC operon is not repressed by isoleucine; and therefore, these strains are very efficient for threonine production. Inactivation of the tdh gene encoding threonine dehydrogenase results in prevention of threonine degradation. The genetic determinant of saccharose assimilation (scrKYABR genes) was transferred to said strain. To increase expression of the genes controlling threonine biosynthesis, the plasmid pVIC40 containing the mutant threonine operon thrA442BC was introduced into the intermediate strain TDH6. The amount of L-threonine which accumulates during fermentation of the strain can be up to 85 g/l.

By optimizing the main biosynthetic pathway of a desired compound, further improvement of L-amino acid producing strains can be accomplished via supplementation of the bacterium with increasing amounts of sugars as a carbon source, for example, glucose. Despite the efficiency of glucose transport by PTS, access to the carbon source in a highly productive strain still may be insufficient.

It is known that the active transport of sugars and other metabolites into bacterial cells is accomplished by several transport systems.

The divergent nagE-BACD operon encodes proteins involved in the uptake and degradation of N-acetylglucosamine (GlcNAc). The nagE gene encodes the N-acetylglucosamine-specific transporter of the phosphotransferase system (PTS) involved in the uptake of GlcNAc and producing intracellular GlcNAc-6-P, while the nagBACD genes encode the two enzymes that degrade GlcNAc-6-P into fructose-6-P (glucosamine-P deaminase (NagB) and N-acetylglucosamine-6-P deacetylase (NagA)), the nag regulon transcriptional regulator (NagC) and a gene of unknown function which is, however, homologous to functionally characterized phosphatases (NagD) (Potsma, P. W., Lengeler, J. W. and Jacobson, G. R. Chapter 72. In Escherichia coli and Salmonella, Second Edition, Editor in Chief: F. C. Neidhardt, ASM Press, Washington D.C., 1996). The Nag repressor, encoded by the nagC gene, binds to two operators, which overlap the nagE and nagB promoters and forms a repression loop of DNA (Plumbridge, J. and Kolb, A. Nucleic Acids Res., 26(5), 1254-60 (1998)). Expression of both nagE and nagB is stimulated by the cAMP/CAP complex, the effect being particularly pronounced for nagE. A binding site for cAMP/CAP is located between the two promoters, i.e. it lies within the DNA which forms a loop. The two arms of the nag regulon are induced in parallel by GlcNAc and glucosamine (GlcN), but the growth and induction characteristics of GlcNAc and GlcN are very different (Plumbridge, J. A., J. Bacteriol., 172, 2728-2735 (1990)). There is little information about the uptake of other sugars by NagE.

The Enzyme II (IINag), encoded by the nagE gene, contains the three functional domains IICBANag fused in one polypeptide. GlcNAc is transported via the EIIC, and phosphate is subsequently transferred from EIIA via the EIIB component onto the C6-hydroxyl group of the imported GlcNAc molecule. EIIA itself is phosphorylated by PEP, and involves the general enzymes of the PTS cascade (EI and HPr). Therefore the NagE can replace Crr (catabolite repression regulator) in Crr-dependent carbohydrate transport and phosphorylation (Potsma, P. W., Lengeler, J. W. and Jacobson, G. R. Chapter 72. In Escherichia coli and Salmonella, Second Edition, Editor in Chief: F. C. Neidhardt, ASM Press, Washington D.C., 1996).

A method to produce glucosamine by fermentation using a microorganism with the increased activity of glucosamine-6-phosphate synthase has been disclosed (U.S. Pat. No. 6,372,457), wherein an additional activity of at least one protein from the group containing N-acetylglucosamine-specific enzyme IINag (N-acetylglucosamine permease) encoded by the nagE gene is decreased.

In many bacteria, components of the phosphoenolpyruvate-dependent sugar phosphotransferase system (PTS) trigger C-regulation by mechanisms known as carbon catabolite repression and inducer exclusion (Potsma, P. W., Lengeler, J. W. and Jacobson, G. R. Chapter 72. and Saier, M. H., Jr., Ramseier, T. M., and Reizer, J. Chapter. In Escherichia coli and Salmonella, Second Edition, Editor in Chief: F. C. Neidhardt, ASM Press, Washington D.C., 1996). One key element of PTS system in Escherichia coli is the enzyme IIA^{Glucose} (IIA^{Glc}) encoded by the crr gene, a small hydrophilic protein which has, in addition to its transport function, a central regulatory role in carbon catabolite repression and inducer exclusion. IIA^{Glc} is phosphorylated by the general PTS proteins, which are histidine-containing phosphoryl carrier protein (HPr) and enzyme I (EI). In turn, it phosphorylates the sugar-specific PTS permeases that catalyse the uptake of glucose, trehalose, and sucrose (Postma, P. W., Lengeler, J. W. & Jacobson, G. R., Microbiol. Rev., 57, 543-594 (1993)). The unphosphorylated IIA^{Glc} inhibits a set of catabolic enzymes and sugar permeases by protein-protein interaction (inducer exclusion). At the same time the cellular cAMP level is low, because dephosphorylated IIA^{Glc} is unable to stimulate adenylate cyclase. Under these conditions the cAMP-dependent catabolite activator protein CAP, which serves as a global activator of many catabolite-controlled genes, remains in a switched off state. IIA^{Glc} further appears to be involved in carbon catabolite repression exerted by non-PTS substrates such as glucose 6-phosphate (Hogema, B. M. et al, Mol. Microbiol., 28, 755-765 (1998)). This could be correlated with the variation of the phosphorylation state of IIA^{Glc}.

Recently, another cellular function of IIA^{Glc} has been proposed. It is suggested that it may be involved in the linkage between carbon metabolism and stress response (Ueguchi, C., Misonou, N. & Mizuno, T., J. Bacteriol., 183, 520-527 (2001)). Mutations in the crr gene, which encodes IIA^{Glc}, exhibit a pleiotropic catabolite repression resistant phenotype.

A method for producing a target substance, such as L-lysine, L-threonine and L-phenylalanine, utilizing a microorganism having mutation in the crr gene was disclosed (EP1254957A2). Process for the fermentative preparation of L-amino acids, in particular L-threonine, using microorganisms of the Enterobacteriaceae family in which one or more of the genes chosen from the group consisting of dps, hns, lrp, pgm, fba, ptsG, ptsH, ptsI, crr, mopB, ahpC and ahpF, or nucleotide sequences which code for these, is (are) attenuated, in particular eliminated, was also disclosed in WO03004662A2 without any working examples.

However, there have been no reports to date of using a bacterium of the Enterobacteriaceae family having an enhanced activity of N-acetylglucosamine PTS permease or a bacterium of the Enterobacteriaceae family having an enhanced activity of N-acetylglucosamine PTS permease and an inactivated crr gene for increasing the production of L-amino acids.

### SUMMARY OF THE INVENTION

Objects of the present invention include enhancing the productivity of L-amino acid-producing strains and providing a method for producing non-aromatic or aromatic L-amino acids using these strains.

The above objects were achieved by finding that increasing the expression of the nagE gene encoding N-acetylglucosamine permease can enhance production of L-amino acids, such as L-threonine, L-lysine, L-histidine, L-phenylalanine, L-arginine, L-tryptophan, and L-glutamic acid.

It is an object of the present invention to provide an L-amino acid-producing bacterium of the Enterobacteriaceae family, wherein said bacterium has been modified to enhance an activity of N-acetylglucosamine permease.

It is a further object of the present invention to provide the bacterium described above, wherein the activity of N-acetylglucosamine permease is enhanced by increasing the expression of a gene which encodes N-acetylglucosamine permease.

It is a further object of the present invention to provide the bacterium described above, wherein the activity of N-acetylglucosamine permease is enhanced by modifying an expression control sequence of the gene encoding N-acetylglucosamine permease so that the gene expression is enhanced or by increasing the copy number of the gene encoding N-acetylglucosamine permease.

It is a further object of the present invention to provide the bacterium described above, wherein said bacterium has been additionally modified to enhance an activity of glucokinase.

It is a further object of the present invention to provide the bacterium described above, where said bacterium has been additionally modified to enhance an activity of xylose isomerase.

It is a further object of the present invention to provide the bacterium described above, wherein said bacterium is selected from the group consisting of the genera Escherichia, Enterobacter, Erwinia, Klebsiella, Pantoea, Providencia, Salmonella, Serratia, Shigella and Morganella.

It is a further object of the present invention to provide the bacterium described above, wherein said gene encodes an N-acetylglucosamine permease selected from the group consisting of:
(A) a protein which comprises the amino acid sequence of SEQ ID NO: 2, and
(B) a variant protein of the amino acid sequence shown in SEQ ID NO: 2, and which has an activity of N-acetylglucosamine permease.

It is a further object of the present invention to provide the bacterium described above, wherein said gene encoding N-acetylglucosamine permease comprises a DNA selected from the group consisting of:
(a) a DNA which comprises a nucleotide sequence of nucleotides 1 to 1947 in SEQ ID NO: 1, and
(b) a DNA which is hybridizable with a nucleotide sequence of nucleotides 1-1947 in SEQ ID NO: 1, or a probe which can be prepared from said nucleotide sequence under stringent conditions, and encodes a protein having an activity of N-acetylglucosamine permease.

It is a further object of the present invention to provide the bacterium described above, wherein said stringent conditions comprise those in which washing is performed at 60° C. at a salt concentration of 1×SSC and 0.1% SDS, for approximately 15 minutes.

It is a further object of the present invention to provide the bacterium described above, wherein said bacterium is an L-threonine producing bacterium.

It is a further object of the present invention to provide the bacterium described above, wherein said bacterium has been additionally modified to enhance expression of one or more of the genes selected from the group consisting of
the mutant thrA gene which codes for aspartokinase homoserine dehydrogenase I and is resistant to feedback inhibition by threonine;
the thrB gene which codes for homoserine kinase;
the thrC gene which codes for threonine synthase;
the rhtA gene which codes for a putative transmembrane protein;
the asd gene which codes for aspartate-β-semialdehyde dehydrogenase; and
the aspC gene which codes for aspartate aminotransferase (aspartate transaminase).

It is a further object of the present invention to provide the bacterium described above, wherein said bacterium has been additionally modified so that the crr gene which codes for catabolite repression regulator is inactivated.

It is a further object of the present invention to provide the bacterium described above, wherein said bacterium is an L-lysine producing bacterium.

It is a further object of the present invention to provide the bacterium described above, wherein said bacterium is an L-histidine producing bacterium.

It is a further object of the present invention to provide the bacterium described above, wherein said bacterium is an L-phenylalanine producing bacterium.

It is a further object of the present invention to provide the bacterium described above, wherein said bacterium is an L-arginine producing bacterium.

It is a further object of the present invention to provide the bacterium described above, wherein said bacterium is an L-tryptophan producing bacterium.

It is a further object of the present invention to provide the bacterium described above, wherein said bacterium is an L-glutamic acid producing bacterium.

It is a further object of the present invention to provide a method for producing an L-amino acid which comprises cultivating the bacterium described above in a culture medium, allowing accumulation of the L-amino acid in the culture medium, and isolating the L-amino acid from the culture medium.

It is a further object of the present invention to provide the method described above, wherein said L-amino acid is L-threonine.

It is a further object of the present invention to provide the method described above, wherein said L-amino acid is L-lysine.

It is a further object of the present invention to provide the method described above, wherein said L-amino acid is L-histidine.

It is a further object of the present invention to provide the method described above, wherein said L-amino acid is L-phenylalanine.

It is a further object of the present invention to provide the method described above, wherein said L-amino acid is L-arginine.

It is a further object of the present invention to provide the method described above, wherein said L-amino acid is L-tryptophan.

It is a further object of the present invention to provide the method described above, wherein said L-amino acid is L-glutamic acid.

### BRIEF DESCRIPTION OF THE DRAWINGS

FIG. 1 shows the structure of the region upstream of the nagE gene in the chromosome of E. Coli and the structure of an integrated DNA fragment containing the cat gene and a P_{tac} promoter.

FIG. 2 shows the alignment of the primary sequences of N-acetylglucosamine permease from Salmonella typhimurium (Stm), Salmonella typhi (St), Escherichia coli (Ec), Shigella flexneri (Sf), Klebsiella pneumoniae (Kp), Yersinis pestis (Yp) and Yersinis pseudotuberculosis (Ypt). The alignment was done by using the PIR Multiple Alignment program (http://pir.georgetown.edu). The identical amino acids are marked by asterisk (*), similar amino acids are marked by colon (:).

### DETAILED DESCRIPTION OF THE PREFERRED EMBODIMENTS

In the present invention, “L-amino acid-producing bacterium” means a bacterium which has an ability to cause accumulation of an L-amino acid in a medium when the bacterium is cultured in the medium. The L-amino acid-producing ability may be imparted or enhanced by breeding. The phrase “L-amino acid-producing bacterium” as used herein also means a bacterium which is able to produce and cause accumulation of an L-amino acid in a culture medium in amount larger than a wild-type or parental strain of E. coli, such as E. coli K-12, and preferably means that the microorganism is able to cause accumulation in a medium of an amount not less than 0.5 g/L, more preferably not less than 1.0 g/L of the target L-amino acid. “L-amino acids” include L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. L-threonine, L-lysine, L-histidine, L-phenylalanine, L-arginine, L-tryptophan, and L-glutamic acid are particularly preferred.

The Enterobacteriaceae family includes bacteria belonging to the genera Escherichia, Enterobacter, Erwinia, Klebsiella, Pantoea, Providencia, Salmonella, Serratia, Shigella, Morganella, etc. Specifically, bacteria classified into the Enterobacteriaceae family according to the taxonomy used in the NCBI (National Center for Biotechnology Information) database (http://www.ncbi.nlm.nih.gov/htbinpost/Taxonomy/wgetorg?mode=Tree&id=1236&lvl=3&keep=1&srchmode=1&unlock) can be used. A bacterium belonging to the genus Escherichia or Pantoea is preferred.

The phrase “a bacterium belonging to the genus Escherichia” means that the bacterium is classified in the genus Escherichia according to the classification known to a person skilled in the art of microbiology. Examples of a bacterium belonging to the genus Escherichia as used in the present invention include, but are not limited to, Escherichia coli (E. coli).

The bacterium belonging to the genus Escherichia that can be used in the present invention is not particularly limited, however for example, bacteria described by Neidhardt, F. C. et al. (Escherichia coli and Salmonella typhimurium, American Society for Microbiology, Washington D.C., 1208, Table 1) are encompassed by the present invention.

The phrase “a bacterium belonging to the genus Pantoea” means that the bacterium is classified into the genus Pantoea according to the classification known to a person skilled in the art of microbiology. Some species of Enterobacter agglomerans have been recently re-classified into Pantoea agglomerans, Pantoea ananatis, Pantoea stewartii, or the like, based on the nucleotide sequence analysis of 16S rRNA etc. (Int. J. Syst. Bacteriol., 43, 162-173 (1993)).

The bacterium of the present invention encompasses a strain of the Enterobacteriaceae family which has an ability to produce an L-amino acid and has been modified to enhance an activity of N-acetylglucosamine permease. In addition, the bacterium of the present invention encompasses a strain of the Enterobacteriaceae family which has an ability to produce an L-amino acid and does not have a native activity of N-acetylglucosamine permease, and has been transformed with a DNA fragment encoding N-acetylglucosamine permease so that the N-acetylglucosamine permease encoded by the DNA fragment is expressed.

The phrase “activity of N-acetylglucosamine permease” means an activity of transporting sugars, such as N-acetylglucosamine and glucose, into the cell. Activity of N-acetylglucosamine permease can be detected and measured by, for example, complementation of growth delay of crr mutants as described by Vogler A. P. and Lengeler J. W. (Mol. Gen. Genet., 213(1), 175-8 (1988)).

The phrase “modified to enhance an activity of N-acetylglucosamine permease” means that the activity per cell is higher as compared to that of a non-modified strain, for example, a wild-type strain. Examples of such modifications include increasing the number of N-acetylglucosamine permease molecules per cell, increasing the specific activity per N-acetylglucosamine permease molecule, and so forth. Furthermore, a wild-type strain that may be used for comparison purposes includes, for example, Escherichia coli K-12. In the present invention, the amount of accumulated L-amino acid, for example, L-threonine, L-lysine, L-histidine, L-phenylalanine, L-arginine, L-tryptophan, or L-glutamic acid, can be increased in a culture medium as a result of enhancing the intracellular activity of N-acetylglucosamine permease.

Enhancing N-acetylglucosamine permease activity in a bacterial cell can be attained by increasing the expression of the nagE gene encoding N-acetylglucosamine permease. Any nagE gene derived from bacteria belonging to the genus Escherichia, as well as any nagE gene derived from other bacteria, such as bacteria belonging to the genus Bacillus, Klebsiella, Pantoea, Salmonella, or Shigella, may be used as the N-acetylglucosamine permease gene of the present invention. nagE genes derived from bacteria belonging to the genus Escherichia are preferred.

The phrase “increasing the expression of the gene” means that the expression of the gene is higher than that of a non-modified strain, for example, a wild-type strain. Examples of such modification include increasing the copy number of gene(s) per cell, increasing the expression level of the gene(s), and so forth. The quantity of the copy number of a gene is measured, for example, by restricting the chromosomal DNA followed by Southern blotting using a probe based on the gene sequence, fluorescence in situ hybridization (FISH), and the like. The level of gene expression can be measured by various known methods including Northern blotting, quantitative RT-PCR, and the like. Furthermore, wild-type strains that can act as a control includes, for example, Escherichia coli K-12 or Pantoea ananatis FERM BP-6614 (WO2004099426, AU2004236516A1). As a result of enhancing the intracellular activity of N-acetylglucosamine permease, L-amino acid accumulation, for example L-threonine, L-lysine, L-histidine, L-phenylalanine, L-tryptophan or L-glutamic acid accumulation in a medium is observed.

The nagE gene which encodes N-acetylglucosamine permease from Escherichia coli has been elucidated (nucleotide numbers complement to numbers 703167 to 705113 in the sequence of GenBank accession NC_{—}000913.2, gi: 16128655). The nagE gene is located between nagB and glnS genes on the chromosome of E. coli K-12. Other nagE genes which encode N-acetylglucosamine permeases have also been elucidated: nagE gene from Shigella flexneri (nucleotide numbers complement to numbers 642063 to 644009 in the sequence of GenBank accession NC_{—}004741.1, gi: 30062146); nagE gene from Salmonella enterica (nucleotide numbers complement to numbers 2257881 to 2259833 in the sequence of GenBank accession NC_{—}004631.1; gi: 29142598), nagE from Yersinia pestis (nucleotide numbers complement to numbers 1163370 to 1165403 in the sequence of GenBank accession NC_{—}005810.1; gi: 45440916), nagE gene from Lactobacillus plantarum (nucleotide numbers 2642875 to 2644863 in the sequence of GenBank accession NC_{—}004567.1; gi:28379408), and the like. An example of the nagE gene from Escherichia coli is represented by SEQ ID NO. 1. The amino acid sequence encoded by the nagE gene is represented by SEQ ID NO: 2.

Upon being transported into the cell, glucose is phosphorylated by glucokinase, which is encoded by the glk gene. So, it is also desirable to modify the bacterium to have enhanced activity of glucokinase. The glk gene which encodes glucokinase of Escherichia coli has been elucidated (nucleotide numbers 2506481 to 2507446 in the sequence of GenBank accession NC_{—}000913.1, gi:16127994). The glk gene is located between the b2387 and the b2389 ORFs on the chromosome of E. coli K-12.

Under appropriate conditions, the xylose isomerase encoded by the xylA gene also efficiently catalyzes the conversion of D-glucose to D-fructose (Wovcha, M. G. et al, Appl Environ Microbiol. 45(4): 1402-4 (1983)). So, it is also desirable to modify the bacterium to have an enhanced activity of xylose isomerase. The xylA gene which encodes xylose isomerase of Escherichia coli has been elucidated (nucleotide numbers 3728788 to 3727466 in the sequence of GenBank accession NC_{—}000913.2, gi: 49175990). The xylA gene is located between xylB and xylF genes on the chromosome of E. coli K-12.

Therefore, nagE, glk and xylA genes can be obtained by PCR (polymerase chain reaction; refer to White, T. J. et al., Trends Genet., 5, 185 (1989)) utilizing primers prepared based on the known nucleotide sequence of the gene. Genes coding for N-acetylglucosamine permease from other microorganisms can be obtained in a similar manner.

The nagE gene derived from Escherichia coli is exemplified by a DNA which encodes the following protein (A) or (B):

(A) a protein which has the amino acid sequence shown in SEQ ID NO: 2, or

(B) a variant protein of the amino acid sequence shown in SEQ ID NO: 2, which has an activity of N-acetylglucosamine permease.

The phrase “variant protein” as used in the present invention means a protein which has changes in the sequence, whether they are deletions, insertions, additions, or substitutions of amino acids, but still maintains the desired activity at a useful level, for example, useful for the enhanced production of an L-amino acid. The number of changes in the variant protein depends on the position or the type of amino acid residue in the three dimensional structure of the protein. The number of changes may be 1 to 30, preferably 1 to 15, and more preferably 1 to 5 for the protein (A). These changes in the variants can occur in regions of the protein which are not critical for the function of the protein. This is because some amino acids have high homology to one another so the three dimensional structure or activity is not affected by such a change. These changes in the variant protein can occur in regions of the protein which are not critical for the function of the protein. Therefore, the protein variant (B) may be one which has a homology of not less than 70%, preferably not less than 80%, and more preferably not less than 90%, and most preferably not less than 95% with respect to the entire amino acid sequence of N-acetylglucosamine permease shown in SEQ ID NO. 2, as long as the activity of N-acetylglucosamine permease is maintained. Homology between two amino acid sequences can be determined using the well-known methods, for example, the computer program BLAST 2.0, which calculates three parameters: score, identity and similarity.

The substitution, deletion, insertion, or addition of one or several amino acid residues should be conservative mutation(s) so that the activity is maintained. The representative conservative mutation is a conservative substitution. Examples of conservative substitutions include substitution of Ser or Thr for Ala, substitution of Gln, H is or Lys for Arg, substitution of Glu, Gln, Lys, H is or Asp for Asn, substitution of Asn, Glu or Gln for Asp, substitution of Ser or Ala for Cys, substitution of Asn, Glu, Lys, H is, Asp or Arg for Gln, substitution of Asn, Gln, Lys or Asp for Glu, substitution of Pro for Gly, substitution of Asn, Lys, Gln, Arg or Tyr for H is, substitution of Leu, Met, Val or Phe for Ile, substitution of Ile, Met, Val or Phe for Leu, substitution of Asn, Glu, Gln, H is or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of H is, Phe or Trp for Tyr, and substitution of Met, Ile or Leu for Val.

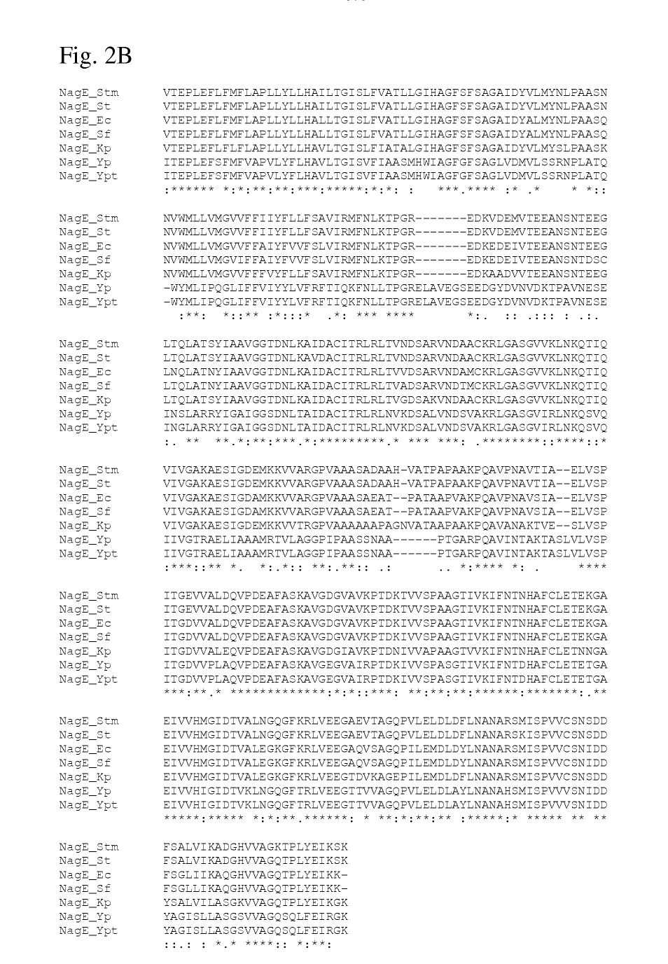

Data comparing the primary sequences of N-acetylglucosamine permease from Salmonella typhimurium (Stm, SEQ ID NO: 25), Salmonella typhi (St, SEQ ID NO: 26), Escherichia coli (Ec, SEQ ID NO: 2), Shigella flexneri (Sf, SEQ ID NO: 27), Klebsiella pneumoniae (Kp, SEQ ID NO: 28), Yersinis pestis (Yp, SEQ ID NO: 29), and Yersinis pseudotuberculosis (Ypt, SEQ ID NO: 30) show a high level of homology among these proteins (see FIG. 2). From this point of view, substitutions or deletions of the amino acid residues which are identical (marked by asterisk) in all the above-mentioned proteins could be crucial for their function. It is possible to substitute similar (marked by colon) amino acids residues by the similar amino acid residues without deterioration of the protein activity. But modifications of other non-conserved amino acid residues may not lead to alteration of the activity of N-acetylglucosamine permease.

The DNA, which encodes substantially the same protein as the N-acetylglucosamine permease described above, may be obtained, for example, by modifying the nucleotide sequence of DNA encoding N-acetylglucosamine permease (SEQ ID NO: 1), for example, by means of the site-directed mutagenesis method so that one or more amino acid residues at a specified site involve deletion, substitution, insertion, or addition. DNA modified as described above may be obtained by conventionally known mutation treatments. Such treatments include hydroxylamine treatment of the DNA encoding proteins of present invention, or treatment of the bacterium containing the DNA with UV irradiation or a reagent such as N-methyl-N′-nitro-N-nitrosoguanidine or nitrous acid.

A DNA encoding substantially the same protein as N-acetylglucosamine permease can be obtained by expressing DNA having a mutation as described above in an appropriate cell, and investigating the activity of any expressed product. A DNA encoding substantially the same protein as N-acetylglucosamine permease can also be obtained by isolating a DNA that is hybridizable with a probe having a nucleotide sequence which contains, for example, the nucleotide sequence shown as SEQ ID NO: 1, under the stringent conditions, and encodes a protein having the N-acetylglucosamine permease activity. The “stringent conditions” referred to herein are conditions under which so-called specific hybrids are formed, and non-specific hybrids are not formed. For example, stringent conditions can be exemplified by conditions under which DNAs having high homology, for example, DNAs having homology of not less than 50%, preferably not less than 60%, more preferably not less than 60%, more preferably not less than 70%, further preferably not less than 80%, and still more preferably not less than 90%, and most preferably not less than 95% are able to hybridize with each other, but DNAs having homology lower than the above are not able to hybridize with each other. Alternatively, stringent conditions may be exemplified by conditions under which DNA is able to hybridize at a salt concentration equivalent to ordinary washing conditions in Southern hybridization, i.e., 1×SSC, 0.1% SDS, preferably 0.1×SSC, 0.1% SDS, at 60° C. Duration of washing depends on the type of membrane used for blotting and, as a rule, what is recommended by the manufacturer. For example, recommended duration of washing, for example for the Hybond™ N+ nylon membrane (Amersham), under stringent conditions is approximately 15 minutes. Preferably, washing may be performed 2 to 3 times.

A partial sequence of the nucleotide sequence of SEQ ID NO: 1 can also be used as a probe. Probes may be prepared by PCR using primers based on the nucleotide sequence of SEQ ID NO: 1, and a DNA fragment containing the nucleotide sequence of SEQ ID NO: 1 as a template. When a DNA fragment having a length of about 300 bp is used as the probe, the hybridization conditions for washing include, for example, 50° C., 2×SSC and 0.1% SDS.

The substitution, deletion, insertion, or addition of nucleotides as described above also includes a mutation which naturally occurs (mutant or variant), for example, due to variety in the species or genus of bacterium, and which contains the N-acetylglucosamine permease.

“Transformation of a bacterium with DNA encoding a protein” means introduction of the DNA into a bacterium, for example, by conventional methods. Transformation of this DNA will result in an increase in expression of the gene encoding the protein of present invention, and will enhance the activity of the protein in the bacterial cell. Methods of transformation include any known methods that have hitherto been reported. For example, a method of treating recipient cells with calcium chloride so as to increase permeability of the cells to DNA has been reported for Escherichia coli K-12 (Mandel, M. and Higa, A., J. Mol. Biol., 53, 159 (1970)) and may be used.

Methods of gene expression enhancement include increasing the gene copy number. Introducing a gene into a vector that is able to function in a bacterium of the Enterobacteriaceae family increases the copy number of the gene. Preferably, low copy vectors are used. Examples of low-copy vectors include but are not limited to pSC101, pMW118, pMW119, and the like. The term “low copy vector” is used for vectors, the copy number of which is up to 5 copies per cell.

Enhancement of gene expression may also be achieved by introduction of multiple copies of the gene into a bacterial chromosome by, for example, a method of homologous recombination, Mu integration, or the like. For example, one act of Mu integration allows introduction of up to 3 copies of the gene into a bacterial chromosome.

Increasing the copy number of the N-acetylglucosamine permease gene can also be achieved by introducing multiple copies of the N-acetylglucosamine permease gene into the chromosomal DNA of the bacterium. In order to introduce multiple copies of the gene into a bacterial chromosome, homologous recombination is carried out using a sequence whose multiple copies exist as targets in the chromosomal DNA. Sequences having multiple copies in the chromosomal DNA include, but are not limited to repetitive DNA, or inverted repeats existing at the end of a transposable element. Also, as disclosed in U.S. Pat. No. 5,595,889, it is possible to incorporate the N-acetylglucosamine permease gene into a transposon, and allow it to be transferred to introduce multiple copies of the gene into the chromosomal DNA.

Enhancing gene expression may also be achieved by placing the DNA of the present invention under the control of a potent promoter. For example, the P_{tac} promoter, the lac promoter, the trp promoter, the trc promoter, the PR, or the P_{L} promoters of lambda phage are all known to be potent promoters. The use of a potent promoter can be combined with multiplication of gene copies.

Alternatively, the effect of a promoter can be enhanced by, for example, introducing a mutation into the promoter to increase the transcription level of a gene located downstream of the promoter. Furthermore, it is known that substitution of several nucleotides in the spacer between ribosome binding site (RBS) and the start codon, especially the sequences immediately upstream of the start codon, profoundly affect the mRNA translatability. For example, a 20-fold range in the expression levels was found, depending on the nature of the three nucleotides preceding the start codon (Gold et al., Annu. Rev. Microbiol., 35, 365-403, 1981; Hui et al., EMBO J., 3, 623-629, 1984). Previously, it was shown that the rhtA23 mutation is an A-for-G substitution at the −1 position relative to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457). Therefore, it may be suggested that the rhtA23 mutation enhances the rhtA gene expression and, as a consequence, increases the resistance to threonine, homoserine and some other substances transported out of cells.

Moreover, it is also possible to introduce a nucleotide substitution into a promoter region of the N-acetylglucosamine permease gene on the bacterial chromosome, which results in stronger promoter function. The alteration of the expression control sequence can be performed, for example, in the same manner as the gene substitution using a temperature-sensitive plasmid, as disclosed in WO 00/18935 and JP 1-215280 A.

Methods for preparation of plasmid DNA include, but are not limited to digestion and ligation of DNA, transformation, selection of an oligonucleotide as a primer and the like, or other methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

The above-described techniques and guidance for enhancing an activity of N-acetylglucosamine permease are similarly applied to enhancing activities of xylose isomerase and glucokinase.

The bacterium of the present invention can be obtained by introduction of the aforementioned DNAs into bacterium which inherently has the ability to produce L-amino acid. Alternatively, the bacterium of the present invention can be obtained by imparting an ability to produce L-amino acid to the bacterium already containing the DNAs.

L-Threonine Producing Bacteria

Examples of parent strains for deriving the L-threonine producing bacteria of the present invention include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A), and the like.

The strain TDH-6 is deficient in the thrC gene, as well as being sucrose-assimilative, and the ilvA gene has a leaky mutation. This strain also has a mutation in the rhtA gene, which imparts resistance to high concentrations of threonine or homoserine. The strain B-3996 contains the plasmid pVIC40 which was obtained by inserting into RSF1010-derived vector a thrA*BC operon which includes a mutant thrA gene. This mutant thrA gene encodes aspartokinase homoserine dehydrogenase I which has substantially desensitized feedback inhibition by threonine. The strain B-3996 was deposited on Nov. 19, 1987 in the All-Union Scientific Center of Antibiotics (Nagatinskaya Street 3-A, 113105 Moscow, Russian Federation) under the accession number RIA 1867. The strain was also deposited in the Russian National Collection of Industrial Microorganisms (VKPM) (Russia, 117545 Moscow 1, Dorozhny proezd 1) on Apr. 7, 1987 under the accession number B-3996.

Preferably, the bacterium of the present invention is additionally modified to enhance expression of one or more of the following genes:
the mutant thrA gene which codes for aspartokinase homoserine dehydrogenase I resistant to feed back inhibition by threonine;
the thrB gene which codes for homoserine kinase;
the thrC gene which codes for threonine synthase;
the rhtA gene which codes for a putative transmembrane protein;
the asd gene which codes for aspartate-β-semialdehyde dehydrogenase; and
the aspC gene which codes for aspartate aminotransferase (aspartate transaminase);

The thrA gene which encodes aspartokinase homoserine dehydrogenase I of Escherichia coli has been elucidated (nucleotide positions 337 to 2799, GenBank accession NC_{—}000913.2, gi: 49175990). The thrA gene is located between thrL and thrB genes on the chromosome of E. coli K-12. The thrB gene which encodes homoserine kinase of Escherichia coli has been elucidated (nucleotide positions 2801 to 3733, GenBank accession NC_{—}000913.2, gi: 49175990). The thrB gene is located between thrA and thrC genes on the chromosome of E. coli K-12. The thrC gene which encodes threonine synthase of Escherichia coli has been elucidated (nucleotide positions 3734 to 5020, GenBank accession NC_{—}000913.2, gi: 49175990). The thrC gene is located between thrB gene and yaaX opened reading frame on the chromosome of E. coli K-12. All three genes are functioning as one threonine operon.

A mutant thrA gene which codes for aspartokinase homoserine dehydrogenase I resistant to feed back inhibition by threonine, as well as, the thrB and thrC genes can be obtained as one operon from well-known plasmid pVIC40 which is presented in the threonine producing E. coli strain VKPM B-3996. Plasmid pVIC40 is described in detail in U.S. Pat. No. 5,705,371.

The rhtA gene exists at 18 min on E. coli chromosome close to the glnHPQ operon, which encodes components of the glutamine transport system. The rhtA gene is identical to ORF1 (ybiF gene, numbers 764 to 1651 in the GenBank accession number AAA218541, gi:440181) and located between the pexB and ompX genes. The unit expressing a protein encoded by the ORF1 has been designated the rhtA gene (rht: resistance to homoserine and threonine) gene. Also, it was revealed that the rhtA23 mutation is an A-for-G substitution at position −1 with respect to the ATG start codon (ABSTRACTS of the 17^{th} International Congress of Biochemistry and Molecular Biology in conjugation with the Annual Meeting of the American Society for Biochemistry and Molecular Biology, San Francisco, Calif., Aug. 24-29, 1997, abstract No. 457, EP 1013765 A).

The asd gene of E. coli has already been elucidated (nucleotide positions 3572511 to 3571408, GenBank accession NC_{—}000913.1, gi:16131307), and can be obtained by PCR (polymerase chain reaction; refer to White, T. J. et al., Trends Genet., 5, 185 (1989)), utilizing primers prepared based on the nucleotide sequence of the gene. The asd genes of other microorganisms can be obtained in a similar manner.

Also, the aspC gene of E. Coli has already been elucidated (nucleotide positions 983742 to 984932, GenBank accession NC_{—}000913.1, gi:16128895), and can be obtained by PCR. The aspC genes of other microorganisms can be obtained in a similar manner. Another example of a parent strain for deriving the L-threonine producing bacteria of the present invention include the E. coli strain MG1655 Δtdh::rhtA* described in Example 1.

More preferably, the bacterium of the present invention is further modified so that the crr gene which codes for catabolite repression regulator is inactivated in addition to enhancement of expression of the mutant thrA gene, the thrB gene, the thrC gene, the rhtA gene, the asd gene, and the aspC gene.

The crr gene which codes for catabolite repression regulator of E. coli has been elucidated (nucleotide positions 2533856 to 2534365, GenBank accession NC_{—}000913.2, gi: 49175990). The crr gene is located between the ptsI and pdxK genes on the chromosome of E. coli K-12.

The phrase “the crr gene is inactivated” means that the target gene is modified so that the modified gene encodes a mutant protein with a decreased activity, or encodes a completely inactive protein. It is also possible that the modified DNA region is unable to provide natural expression of the gene due to the deletion of a part of the gene, the shifting of the reading frame of the gene, the introduction of missense/nonsense mutation, or the modification of an adjacent region of the gene, including sequences controlling gene expression, such as promoter(s), enhancer(s), attenuator(s), ribosome binding site(s), etc.

Inactivation of the gene can be performed by conventional methods, such as a mutagenesis treatment using UV irradiation or nitrosoguanidine (N-methyl-N′-nitro-N-nitrosoguanidine), a site-directed mutagenesis, a gene disruption using homologous recombination or/and an insertion-deletion mutagenesis (Yu, D. et al., Proc. Natl. Acad. Sci. USA, 2000, 97:12: 5978-83; Datsenko K. A. and Wanner B. L., Proc. Natl. Acad. Sci. USA, 2000, 97:12: 6640-45), also called “Red-driven integration”.

L-Lysine Producing Bacteria

Examples of L-lysine producing bacteria belonging to the genus Escherichia include mutants having resistance to an L-lysine analogue. The L-lysine analogue inhibits growth of bacteria belonging to the genus Escherichia, but this inhibition is fully or partially desensitized when L-lysine coexists in a medium. Examples of the L-lysine analogue include, but are not limited to, oxalysine, lysine hydroxamate, S-(2-aminoethyl)-L-cysteine (AEC), γ-methyllysine, α-chlorocaprolactam, and so forth. Mutants having resistance to these lysine analogues can be obtained by subjecting bacteria belonging to the genus Escherichia to a conventional artificial mutagenesis treatment. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The strain WC196 may be used as an L-lysine producing bacterium of Escherichia coli. This bacterial strain was bred by conferring AEC resistance to the strain W3110, which was derived from Escherichia coli K-12. The resulting strain was designated as Escherichia coli AJ13069, and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was converted to an international deposit under the provisions of the Budapest Treaty on Sep. 29, 1995, and received an accession number of FERM BP-5252 (U.S. Pat. No. 5,827,698).

Examples of parent strains for deriving L-lysine-producing bacteria of the present invention also include strains in which expression of one or more genes encoding an L-lysine biosynthetic enzyme are enhanced. Examples of the enzymes involved in L-lysine biosynthesis include, but are not limited to, dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), nicotinamide adenine dinucleotide transhydrogenase (pntAB), and aspartase (aspA) (EP 1253195 A).

Examples of parent strains for deriving L-lysine-producing bacteria of the present invention also include strains having decreased or eliminated activity of an enzyme that catalyzes a reaction for generating a compound other than L-lysine by branching off from the biosynthetic pathway of L-lysine. Examples of the enzymes that catalyze a reaction for generating a compound other than L-lysine by branching off from the biosynthetic pathway of L-lysine include homoserine dehydrogenase and lysine decarboxylase (U.S. Pat. No. 5,827,698).

L-Histidine Producing Bacteria

Examples of parent strains for deriving L-histidine producing bacteria of the present invention include, but are not limited to, strains belonging to the genus Escherichia, such as E. coli strain 24 (VKPM B-5945, RU2003677); E. coli strain 80 (VKPM B-7270, RU2119536); E. coli NRRL B-12116-B12121 (U.S. Pat. No. 4,388,405); E. coli H-9342 (FERM BP-6675) and H-9343 (FERM BP-6676) (U.S. Pat. No. 6,344,347); E. coli H-9341 (FERM BP-6674) (EP1085087); E. coli A180/pFM201 (U.S. Pat. No. 6,258,554), and the like.

Examples of parent strains for deriving L-histidine-producing bacteria of the present invention also include strains in which expression of one or more genes encoding an L-histidine biosynthetic enzyme are enhanced. Examples of the L-histidine-biosynthetic enzymes include ATP phosphoribosyltransferase (hisG), phosphoribosyl AMP cyclohydrolase (hisI), phosphoribosyl-ATP pyrophosphohydrolase (hisIE), phosphoribosylformimino-5-aminoimidazole carboxamide ribotide isomerase (hisA), amidotransferase (hisH), histidinol phosphate aminotransferase (hisC), histidinol phosphatase (hisB), histidinol dehydrogenase (hisD), and so forth.

It is known that the genes encoding the L-histidine biosynthetic enzyme (hisG, hisBHAFI) are inhibited by L-histidine, and therefore an L-histidine-producing ability can also be efficiently enhanced by introducing a mutation conferring resistance to the feedback inhibition into ATP phosphoribosyltransferase (hisG) (Russian Patent Nos. 2003677 and 2119536).

Specific examples of strains having an L-histidine-producing ability include E. coli FERM-P 5038 and 5048 which have been introduced with a vector carrying a DNA encoding an L-histidine-biosynthetic enzyme (JP 56-005099 A), E. coli strains introduced with rht, a gene for an amino acid-export (EP1016710A), E. coli 80 strain imparted with sulfaguanidine, DL-1,2,4-triazole-3-alanine, and streptomycin-resistance (VKPM B-7270, Russian Patent No. 2119536), and so forth.

L-Phenylalanine Producing Bacteria

Examples of parent strains for deriving L-phenylalanine producing strains of the present invention include, but are not limited to, strains belonging to the genus Escherichia, such as E. coli AJ12739 (tyrA::Tn10, tyrR) (VKPM B-8197); E. coli HW1089 (ATCC 55371) harboring pheA34 gene (U.S. Pat. No. 5,354,672); E. coli MWEC101-b (KR8903681); E. coli NRRL B-12141, NRRL B-12145, NRRL B-12146 and NRRL B-12147 (U.S. Pat. No. 4,407,952), and the like. Also, as a parent strain which can be enhanced in activity of the protein of the present invention, L-phenylalanine-producing bacteria belonging to the genus Escherichia, E. coli K-12 [W3110 (tyrA)/pPHAB] (FERM BP-3566), E. coli K-12 [W3110 (tyrA)/pPHAD] (FERM BP-12659), E. coli K-12 [W3110 (tyrA)/pPHATerm] (FERM BP-12662) and E. coli K-12 [W3110 (tyrA)/pBR-aroG4, PACMAB] named AJ 12604 (FERM BP-3579) may be used (EP 488424 B1). Furthermore, L-phenylalanine producing bacteria belonging to the genus Escherichia with an enhanced activity of a protein encoded by the yedA gene or the yddG gene may also be used (U.S. Patent Applications 2003/0148473 A1 and 2003/0157667 A1).

L-Arginine Producing Bacteria

Examples of parent strains for deriving L-arginine producing bacteria of the present invention include, but are not limited to, strains belonging to the genus Escherichia, such as E. coli mutants having resistance to α-methylmethionine, p-fluorophenylalanine, D-arginine, arginine hydroxamate, S-(2-aminoethyl)-cysteine, α-methylserine, β-2-thienylalanine, or sulfaguanidine (JP 56-106598 A); an L-arginine-producing strain into which the argA gene encoding N-acetylglutamate synthetase is introduced (EP1170361A1); strains 237 (VKPM B-7925) and 382 (VKPM B-7926) described in EP1170358A1, and the like.

Examples of parent strains for deriving L-arginine producing bacteria of the present invention also include strains in which expression of one or more genes encoding an L-arginine biosynthetic enzyme are enhanced. Examples of the L-arginine biosynthetic enzymes include N-acetylglutamyl phosphate reductase (argC), ornithine acetyl transferase (argJ), N-acetylglutamate kinase (argB), acetylornithine transaminase (argD), ornithine carbamoyl transferase (argF), argininosuccinic acid synthetase (argG), argininosuccinic acid lyase (argH), and carbamoyl phosphate synthetase. These arginine biosynthetic genes exist on the Arg operon (argCJBDFRGH), and are regulated by an arginine repressor encoded by argR (J Bacteriol. 2002 December; 184(23):6602-14). Therefore, disruption of the arginine repressor results in an increase in the expression of the Arg operon, thus enhancing the activities of the L-arginine-producing enzymes (U.S. Patent Application 2002/0045223 A1).

L-Tryptophan Producing Bacteria

Examples of parent strains for deriving the L-tryptophan-producing bacteria of the present invention include, but are not limited to, strains belonging to the genus Escherichia, such as E. coli JP4735/pMU3028 (DSM10122) and JP6015/pMU91 (DSM10123) deficient in the tryptophanyl-tRNA synthetase encoded by mutant trpS gene (U.S. Pat. No. 5,756,345); E. coli SV164 (pGH5) having the serA allele encoding phosphoglycerate dehydrogenase free from feedback inhibition by serine and the trpE allele encoding anthranilate synthase free from feedback inhibition by tryptophan (U.S. Pat. No. 6,180,373); E. coli AGX17 (pGX44) (NRRL B-12263) and AGX6(pGX50)aroP (NRRL B-12264) deficient in the enzyme tryptophanase (U.S. Pat. No. 4,371,614); E. coli strain AGX17/pGX50,pACKG4-pps in which a phosphoenolpyruvate-producing ability is enhanced (WO9708333, U.S. Pat. No. 6,319,696), and the like, may be used.

Previously, it was identified that the yddG gene, which encodes a membrane protein which is not involved in a biosynthetic pathway of any L-amino acid, and imparts a microorganism resistance to L-phenylalanine and several amino acid analogues when the wild-type allele of the gene was amplified on a multi-copy vector in the microorganism. Besides, the yddG gene can enhance production of L-phenylalanine or L-tryptophan when additional copies are introduced into the cells of the respective producing strain (WO03044192). So, it is desirable that the L-tryptophan-producing bacterium be further modified to have enhanced expression of the yddG open reading frame.

Examples of parent strains for deriving the L-tryptophan-producing bacteria of the present invention also include strains in which one or more activities of the enzymes selected from anthranilate synthase, phosphoglycerate dehydrogenase, and tryptophan synthase are enhanced. The anthranilate synthase and phosphoglycerate dehydrogenase are both subject to feedback inhibition by L-tryptophan and L-serine, so that a mutation desensitizing the feedback inhibition may be introduced into these enzymes. Specific examples of strains having such a mutation include a E. coli SV164 which harbors desensitized anthranilate synthase and a transformant strain obtained by introducing into the E. coli SV164 the plasmid pGH5 (WO 94/08031), which contains a mutant serA gene encoding feedback-desensitized phosphoglycerate dehydrogenase.

Examples of parent strains for deriving the L-tryptophan-producing bacteria of the present invention also include strains into which the tryptophan operon which contains a gene encoding desensitized anthranilate synthase has been introduced (JP 57-71397 A, JP 62-244382 A, U.S. Pat. No. 4,371,614). Moreover, L-tryptophan-producing ability may be imparted by enhancing expression of a gene which encodes tryptophan synthase, among tryptophan operon (trpBA). The tryptophan synthase consists of α and β subunits which are encoded by trpA and trpB, respectively.

L-Glutamic Acid Producing Bacteria

Examples of parent strains for deriving L-glutamic acid-producing bacteria of the present invention include, but are not limited to, strains such as E. coli VL334thrC⁺ (EP 1172433). E. coli VL334 (VKPM B-1641) is an L-isoleucine and L-threonine auxotrophic strain having mutations in thrC and ilvA genes (U.S. Pat. No. 4,278,765). A wild-type allele of the thrC gene was transferred by the method of general transduction using a bacteriophage P1 grown on wild-type E. coli K12 (VKPM B-7) cells. As a result, an L-isoleucine auxotrophic strain VL334thrC⁺ (VKPM B-8961) was obtained. This strain is able to produce L-glutamic acid.

Examples of parent strains for deriving the L-glutamic acid-producing bacteria of the present invention include, but are not limited to, strains in which expression of one or more genes encoding an L-glutamic acid biosynthetic enzyme are enhanced. Examples of the enzymes involved in L-glutamic acid biosynthesis include glutamate dehydrogenase, glutamine synthetase, glutamate synthetase, isocitrate dehydrogenase, aconitate hydratase, citrate synthase, phosphoenolpyruvate carboxylase, pyruvate carboxylase, pyruvate dehydrogenase, pyruvate kinase, phosphoenolpyruvate synthase, enolase, phosphoglyceromutase, phosphoglycerate kinase, glyceraldehyde-3-phophate dehydrogenase, triose phosphate isomerase, fructose bisphosphate aldolase, phosphofructokinase, and glucose phosphate isomerase.

Examples of strains modified so that expression of the citrate synthetase gene, the phosphoenolpyruvate carboxylase gene, and/or the glutamate dehydrogenase gene is/are enhanced include those disclosed in EP1078989A, EP955368A, and EP952221A.

Examples of parent strains for deriving the L-glutamic acid-producing bacteria of the present invention also include strains having decreased or eliminated activity of an enzyme that catalyzes synthesis of a compound other than L-glutamic acid, and branching off from an L-glutamic acid biosynthesis pathway. Examples of such enzymes include isocitrate lyase, α-ketoglutarate dehydrogenase, phosphotransacetylase, acetate kinase, acetohydroxy acid synthase, acetolactate synthase, formate acetyltransferase, lactate dehydrogenase, and glutamate decarboxylase. Bacteria belonging to the genus Escherichia deficient in the α-ketoglutarate dehydrogenase activity or have a reduced α-ketoglutarate dehydrogenase activity, and methods for obtaining them are described in U.S. Pat. Nos. 5,378,616 and 5,573,945. Specifically, these strains include the following:

E. coli W3110sucA::Kmr

E. coli AJ12624 (FERM BP-3853)

E. coli AJ12628 (FERM BP-3854)

E. coli AJ12949 (FERM BP-4881)

E. coli W3110sucA::Kmr is a strain which is obtained by disrupting the α-ketoglutarate dehydrogenase gene (hereinafter referred to as “sucA gene”) of E. coli W3110. This strain is completely deficient in the α-ketoglutarate dehydrogenase.

Other examples of L-glutamic acid-producing bacterium include those which belong to the genus Escherichia and have resistance to an aspartic acid antimetabolite. These strains can also be deficient in the α-ketoglutarate dehydrogenase activity and include, for example, E. coli AJ13199 (FERM BP-5807) (U.S. Pat. No. 5,908,768), FFRM P-12379, which additionally has a low L-glutamic acid decomposing ability (U.S. Pat. No. 5,393,671); AJ13138 (FERM BP-5565) (U.S. Pat. No. 6,110,714), and the like.

Examples of L-glutamic acid-producing bacteria, include mutant strains belonging to the genus Pantoea which are deficient in the α-ketoglutarate dehydrogenase activity or have a decreased α-ketoglutarate dehydrogenase activity, and can be obtained as described above. Such strains include Pantoea ananatis AJ13356 (U.S. Pat. No. 6,331,419). Pantoea ananatis AJ13356 was deposited at the National Institute of Bioscience and Human-Technology, Agency of Industrial Science and Technology, Ministry of International Trade and Industry (currently, National Institute of Advanced Industrial Science and Technology, International Patent Organism Depositary, Central 6, 1-1, Higashi 1-Chome, Tsukuba-shi, Ibaraki-ken, 305-8566, Japan) on Feb. 19, 1998 and received an accession number of FERM P-16645. It was then converted to an international deposit under the provisions of the Budapest Treaty on Jan. 11, 1999 and received an accession number of FERM BP-6615. Pantoea ananatis AJ13356 is deficient in α-ketoglutarate dehydrogenase activity as a result of disruption of the αKGDH-E1 subunit gene (sucA). The above strain was identified as Enterobacter agglomerans when it was isolated and deposited as the Enterobacter agglomerans AJ13356. However, it was recently re-classified as Pantoea ananatis on the basis of nucleotide sequencing of 16S rRNA and so forth. Although AJ13356 was deposited at the aforementioned depository as Enterobacter agglomerans, for the purposes of this specification, they are described as Pantoea ananatis.

Production of L-Amino Acids

Oxaloacetate (OAA) serves as a substrate for the reaction which results in synthesis of Thr and Lys. OAA results from a reaction of PEP with phosphoenol pyrvate carboxlase (PEPC) functioning as a catalyst. Therefore, elevation of the PEPC concentration in a cell can be very important for fermentative production of these amino acids. When using glucose as the carbon source in fermentation, glucose is internalized by the glucose-phosphontransferase (Glc-PTS) system. This system consumes PEP, and proteins in the PTS are encoded by ptsG and ptsHIcrr. During internalization, one molecule of PEP and one molecule of pyruvate (Pyr) are generated from one molecule of glucose.

An L-threonine-producing strain and an L-lysine-producing strain which have been modified to have an ability to utilize sucrose (Scr-PTS) have higher productivity of these amino acids when cultured in sucrose rather than glucose (EP 1149911 A2). It is believed that three molecules of PEP and one molecule of Pyr are generated from one molecule of sucrose by the Scr-PTS, increasing the ratio of PEP/Pyr, and thereby facilitating the synthesis of Thr and Lys from sucrose. Furthermore, it has been reported that Glc-PTS is subject to several expression controls (Postma P. W. et al., Microbiol Rev., 57(3), 543-94 (1993); Clark B. et al. J. Gen. Microbiol., 96(2), 191-201 (1976); Plumbridge J., Curr. Opin. Microbiol., 5(2), 187-93 (2002); Ryu S. et al., J. Biol. Chem., 270(6):2489-96 (1995)), and hence it is possible that the incorporation of glucose itself can be a rate-limiting step in amino acid fermentation.

Increasing the ratio of PEP/Pyr even more by increasing expression of the nagE gene in a threonine-producing strain, a lysine-producing strain, a histidine-producing strain, a phenylalanine-producing strain, an arginine-producing strain, a tryptophan-producing strain and/or a glutamic acid-producing strain should further increase amino acid production. Because four molecules of PEP are generated from two molecules of glucose, the ratio of PEP/Pyr is expected to be greatly improved. Due to the increased expression of the nagE gene, removal of the expression control glc-PTS is expected.

The method for producing an L-amino acid of the present invention includes the steps of cultivating the bacterium of the present invention in a culture medium, allowing L-amino acid to accumulate in the culture medium, and collecting L-amino acid from the culture medium. Furthermore, the method of present invention includes a method for producing L-threonine, L-lysine, L-histidine, L-phenylalanine, L-arginine, L-tryptophan, or L-glutamic acid, including the steps of cultivating the bacterium of the present invention in a culture medium, allowing L-threonine, L-lysine, L-histidine, L-phenylalanine, L-arginine, L-tryptophan, or L-glutamic acid to accumulate in the culture medium, and collecting L-threonine, L-lysine, L-histidine, L-phenylalanine, L-arginine, L-tryptophan, or L-glutamic acid from the culture medium.

In the present invention, the cultivation, collection, and purification of L-amino acids from the medium and the like may be performed by conventional fermentation methods wherein an L-amino acid is produced using a bacterium.

The culture medium may be either synthetic or natural, so long as the medium includes a carbon source, a nitrogen source, minerals, and if necessary, appropriate amounts of nutrients which the bacterium requires for growth. The carbon source may include various carbohydrates such as glucose and sucrose, and various organic acids. Depending on the mode of assimilation of the bacterium used, alcohols, including ethanol and glycerol may be used. As the nitrogen source, various ammonium salts such as ammonia and ammonium sulfate, other nitrogen compounds such as amines, a natural nitrogen source such as peptone, soybean-hydrolysate, and digested fermentative microorganisms may be used. As minerals, potassium monophosphate, magnesium sulfate, sodium chloride, ferrous sulfate, manganese sulfate, calcium chloride, and the like may be used. As vitamins, thiamine, yeast extract, and the like may be used. Additional nutrients may be added to the medium, if necessary. For example, if the bacterium requires an L-amino acid for growth (L-amino acid auxotrophy), a sufficient amount of the L-amino acid may be added to the cultivation medium.

The cultivation is preferably performed under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to accumulation of the target L-amino acids in the liquid medium.

After cultivation, solids such as cells can be removed from the liquid medium by centrifugation or membrane filtration, and then target L-amino acids can be collected and purified by ion-exchange, concentration, and crystallization methods.

### EXAMPLES

The present invention will be more concretely explained below with reference to the following non-limiting examples.

### Example 1

### Preparation of the E. coli Strain MG1655 Δtdh::rhtA*

The L-threonine producing E. coli strain MG1655 Δtdh, rhtA* (pVIC40) was constructed by inactivation of the native tdh gene in E. coli MG1655 (ATCC700926) using the cat gene followed by introduction of an rhtA23 mutation which confers resistance to high concentrations of threonine (>40 mg/ml) and homoserine (>5 mg/ml). Then, the resulting strain was transformed with plasmid pVIC40 from E. coli VKPM B-3996. Plasmid pVIC40 is described in detail in the U.S. Pat. No. 5,705,371.

To substitute the native tdh gene, a DNA fragment carrying the chloramphenicol resistance marker (Cm^{R}) encoded by the cat gene was integrated into the chromosome of E. coli MG1655 in place of the native gene by the method described by Datsenko K. A. and Wanner B. L. (Proc. Natl. Acad. Sci. USA, 2000, 97, 6640-6645) which is also called “Red-mediated integration” and/or “Red-driven integration”. The recombinant plasmid pKD46 (Datsenko, K. A., Wanner, B. L., Proc. Natl. Acad. Sci. USA, 2000, 97, 6640-6645) with the thermosensitive replicon was used as the donor of the phage λ-derived genes responsible for the Red-mediated recombination system. E. coli BW25113 containing the recombinant plasmid pKD46 can be obtained from the E. coli Genetic Stock Center, Yale University, New Haven, USA, the accession number of which is CGSC7630. The recombinant plasmid pKD46 (Datsenko, K. A., Wanner, B. L., Proc. Natl. Acad. Sci. USA, 2000, 97, 6640-6645) with the thermosensitive replicon was used as the donor of the phage λ-derived genes responsible for the Red-mediated recombination system. E. coli BW25113 containing the recombinant plasmid pKD46 can be obtained from the E. coli Genetic Stock Center, Yale University, New Haven, USA, the accession number of which is CGSC7630.

A DNA fragment containing a Cm^{R} marker encoded by the cat gene was obtained by PCR using the commercially available plasmid pACYC184 (GenBank/EMBL accession number X06403, “Fermentas”, Lithuania) as the template, and primers P1 (SEQ ID NO: 3) and P2 (SEQ ID NO: 4). Primer P1 contains 35 nucleotides homologous to the 5′-region of the tdh gene introduced into the primer for further integration into the bacterial chromosome. Primer P2 contains 32 nucleotides homologous to the 3′-region of the tdh gene introduced into the primer for further integration into the bacterial chromosome.

PCR was provided using the “Gene Amp PCR System 2700” amplificatory (Applied Biosystems). The reaction mixture (total volume—50 μl) consisted of 5 μl of 10×PCR-buffer with 25 mM MgCl₂ (“Fermentas”, Lithuania), 200 μM each of dNTP, 25 pmol each of the exploited primers and 1 U of Taq-polymerase (“Fermentas”, Lithuania). Approximately 5 ng of the plasmid DNA was added in the reaction mixture as a template DNA for the PCR amplification. The temperature profile was the following: initial DNA denaturation for 5 min at 95° C., followed by 25 cycles of denaturation at 95° C. for 30 sec, annealing at 55° C. for 30 sec, elongation at 72° C. for 40 sec; and the final elongation for 5 min at 72° C. Then, the amplified DNA fragment was purified by agarose gel-electrophoresis, extracted using “GenElute Spin Columns” (Sigma, USA), and precipitated by ethanol.

The obtained DNA fragment was used for electroporation and Red-mediated integration into the bacterial chromosome of the E. Coli MG1655/pKD46.

MG1655/pKD46 cells were grown overnight at 30° C. in the liquid LB-medium with addition of ampicillin (100 μg/ml), then diluted 1:100 by the SOB-medium (Yeast extract, 5 g/l; NaCl, 0.5 g/l; Tryptone, 20 g/l; KCl, 2.5 mM; MgCl₂, 10 mM) with addition of ampicillin (100 μg/ml) and L-arabinose (10 mM) (arabinose is used for inducing the plasmid containing the genes of the Red system) and grown at 30° C. to reach the optical density of the bacterial culture OD₆₀₀=0.4-0.7. The grown cells from 10 ml of the bacterial culture were washed 3 times with ice-cold de-ionized water, followed by suspending in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions. Shocked cells were added to 1-ml of SOC medium (Sambrook et al, “Molecular Cloning A Laboratory Manual, Second Edition”, Cold Spring Harbor Laboratory Press (1989)), incubated 2 hours at 37° C., and then were spread onto L-agar containing 25 μg/ml of chloramphenicol. Colonies grown for 24 hours were tested for the presence of Cm^{R} marker instead of the native tdh gene by PCR using primers P3 (SEQ ID NO: 5) and P4 (SEQ ID NO: 6). For this purpose, a freshly isolated colony was suspended in 20 μl water and then 1 μl of obtained suspension was used for PCR. The temperature profile follows: initial DNA denaturation for 5 min at 95° C.; then 30 cycles of denaturation at 95° C. for 30 sec, annealing at 55° C. for 30 sec and elongation at 72° C. for 30 sec; the final elongation for 5 min at 72° C. A few Cm^{R} colonies tested contained the desired 1104 bp DNA fragment, confirming the presence of Cm^{R} marker DNA instead of 1242 bp fragment of tdh gene. One of the obtained strains was cured from the thermosensitive plasmid pKD46 by culturing at 37° C. and the resulting strain was named as E. coli MG1655Δtdh.

Then, the rhtA23 mutation from the strain VL614rhtA23 (Livshits V. A. et al, 2003, Res. Microbiol., 154:123-135) was introduced into obtained strain MG1655 Δtdh resulting in the strain MG1655 Δtdh, rhtA*. The rhtA23 is a mutation which confers resistance to high concentrations of threonine (>40 mg/ml) and homoserine (>5 mg/ml). For that purpose the strain MG1655 Δtdh was infected with phage P1ᵥᵢᵣ grown on the donor strain VL614rhtA23. The transductants were selected on M9 minimal medium containing 8 mg/ml homoserine and 0.4% glucose as the sole carbon source.

### Example 2

### Substitution of the Native Promoter Region of the nagE Gene in E. coli by P_{tac} Promoter

To substitute the native promoter region of the nagE gene, a DNA fragment carrying a modified P_{tac} promoter (with deletion of one nucleotide in LacI-binding site region that led to absence of LacI-dependent repression) and kanamycin resistance marker (Km^{R}) encoded by the kan gene was integrated into the chromosome of E. coli MG1655 Δtdh::rhtA in place of the native promoter region by the method described by Datsenko K. A. and Wanner B. L. (Proc. Natl. Acad. Sci. USA, 2000, 97, 6640-6645) which is also called “Red-mediated integration” and/or “Red-driven integration”.

The modified P_{tac} promoter was obtained by PCR using the commercially available plasmid pK 223-3 (Pharmacia) as a template and primers P5 (SEQ ID NO: 7) and P6 (SEQ ID NO: 8). Primer P5 contains a BglII recognition site at the 3′-end thereof, which is necessary for further joining to the kan gene and primer P6 contains 29 nucleotides homologous to the 3′-region of the P_{tac} promoter (with deletion of C-nucleotide in LacI-binding site region).

A DNA fragment containing a Km^{R} marker encoded by the kan gene was obtained by PCR using the commercially available plasmid pACYC177 (GenBank/EMBL accession number X06402, “Fermentas”, Lithuania) as the template, and primers P7 (SEQ ID NO: 9) and P8 (SEQ ID NO: 10). Primer P7 contains 41 nucleotides homologous to the region located 120 bp upstream of the start codon of the nagE gene introduced into the primer for further integration into the bacterial chromosome and primer P8 contains a BglII recognition site at the 5′-end thereof, which is necessary for further joining to the modified P_{tac} promoter.

PCR was provided using the “Gene Amp PCR System 2700” amplificatory (Applied Biosystems). The reaction mixture (total volume—50 μl) consisted of 5 μl of 10×PCR-buffer with 25 mM MgCl₂ (“Fermentas”, Lithuania), 200 μM each of dNTP, 25 pmol each of the exploited primers and 1 U of Taq-polymerase (“Fermentas”, Lithuania). Approximately 5 ng of the plasmid DNA was added to the reaction mixture as a template DNA for the PCR amplification. The temperature profile was the following: initial DNA denaturation at 95° C. for 5 min, followed by 25 cycles of denaturation at 95° C. for 30 sec, annealing at 55° C. for 30 sec, elongation at 72° C. for 20 sec for P_{tac} promoter and 50 sec for kan gene; and the final elongation for 5 min at 72° C. Then, the amplified DNA fragment was purified by agarose gel-electrophoresis, extracted using “GenElute Spin Columns” (Sigma, USA), and precipitated by ethanol.

Each of the two above-described DNA fragments was treated with BglII restrictase and ligated. The ligation product was amplified by PCR using primers P6 (SEQ ID NO: 8) and P7 (SEQ ID NO: 9) The amplified kan-P_{tac} nagE DNA fragment was purified by agarose gel-electrophoresis, extracted using “GenElute Spin Columns” (Sigma, USA) and precipitated by ethanol. The obtained DNA fragment was used for electroporation and Red-mediated integration into the bacterial chromosome of the E. coli MG1655Δtdh::rhtA/pKD46.

MG1655 Δtdh, rhtA/pKD46 cells were grown overnight at 30° C. in the liquid LB-medium with the addition of ampicillin (100 μg/ml), then diluted 1:100 with the SOB-medium (Yeast extract, 5 g/l; NaCl, 0.5 g/l; Tryptone, 20 g/l; KCl, 2.5 mM; MgCl₂, 10 mM) with the addition of ampicillin (100 μg/ml) and L-arabinose (10 mM) (arabinose is used for inducing the plasmid encoding genes of the Red system) and grown at 30° C. to reach the optical density of the bacterial culture OD₆₀₀=0.4-0.7. Grown cells from 10 ml of the bacterial culture were washed 3 times with ice-cold de-ionized water, followed by suspending in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions.

Shocked cells were added to 1-ml of SOC medium (Sambrook et al, “Molecular Cloning A Laboratory Manual, Second Edition”, Cold Spring Harbor Laboratory Press (1989)), incubated 2 hours at 37° C., and then were spread onto L-agar containing 20 μg/ml of kanamycin.

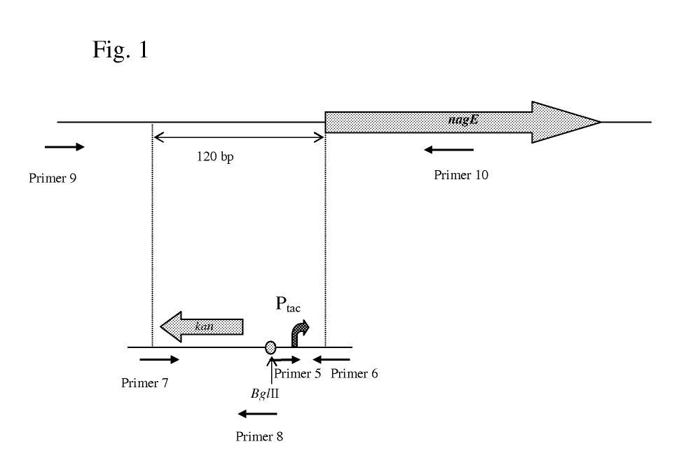

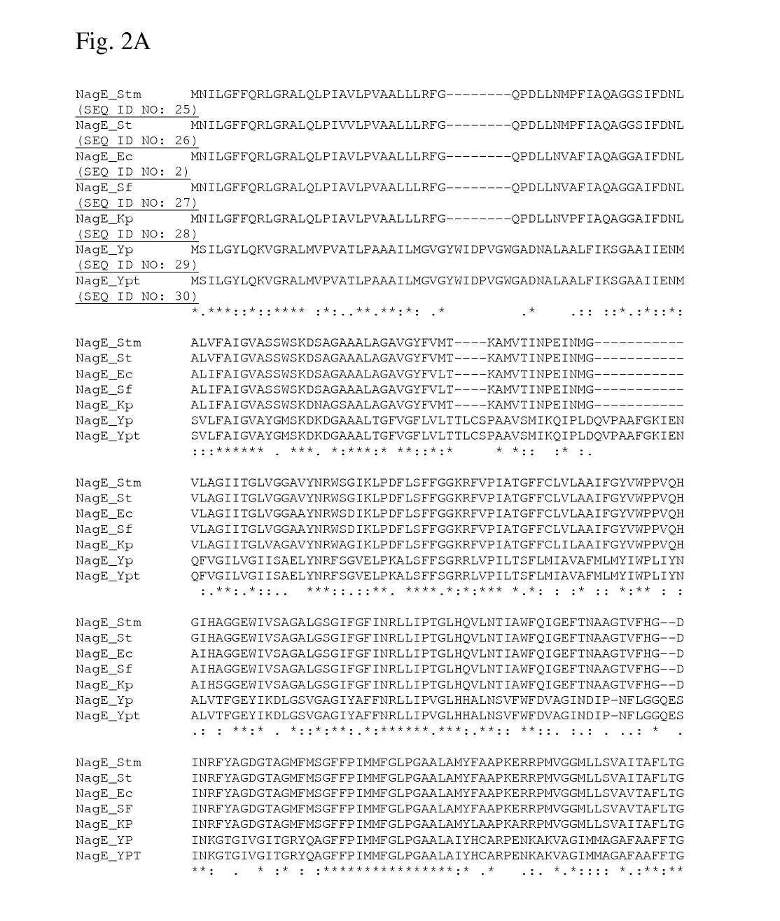

Colonies grown within 24 hours were tested for the presence of Km^{R} marker, instead of the native promoter region of the nagE gene by PCR using primers P9 (SEQ ID NO: 11) and P10 (SEQ ID NO: 12). For this purpose, a freshly isolated colony was suspended in 20 μl water and then 1 μl of obtained suspension was used for PCR. The following temperature profile was used: initial DNA denaturation for 10 min at 95° C.; then 30 cycles of denaturation at 95° C. for 30 sec, annealing at 55° C. for 30 sec and elongation at 72° C. for 1.5 min; the final elongation for 5 min at 72° C. A few Km^{R} colonies tested contained the desired 1700 bp DNA fragment, confirming the presence of Km^{R} marker DNA instead of 600 bp native promoter region of nagE gene (see FIG. 1). One of the obtained strains was cured from the thermosensitive plasmid pKD46 by culturing at 37° C. and the resulting strain was named as E. coli MG1655Δtdh,rhtA*,P_{tac}nagE.

### Example 3

### Effect of Increasing the nagE Gene Expression on L-Threonine Production

To evaluate the effect of enhancing expression of the nagE gene on threonine production, both E. coli strains MG1655Δtdh, rhtA*, P_{tac}nagE and MG1655Δtdh, rhtA* were transformed with plasmid pVIC40.

Then E. coli strains MG1655Δtdh, rhtA*, P_{tac}nagE and MG1655Δtdh, rhtA* were each cultivated at 37° C. for 18 hours in a nutrient broth, and 0.3 ml of each of the obtained cultures was inoculated into 3 ml of fermentation medium having the following composition in a 20×200 mm test tube and cultivated at 37° C. for 24 hours with a rotary shaker.

After cultivation, the accumulated amount of L-threonine in the medium was determined by paper chromatography using the following mobile phase: butanol:acetic acid:water=4:1:1 (v/v). A solution (2%) of ninhydrin in acetone was used as a visualizing reagent. A spot containing L-threonine was cut off, L-threonine was eluted in 0.5% water solution of CdCl₂, and the amount of L-threonine was determined spectrophotometrically at 540 nm. The results of test tube fermentation are shown in Table 1.

The composition of the fermentation medium (g/l) is as follows:

| | |
|---|---|
| Glucose | 40.0 |
| (NH₄)₂SO₄ | 16.0 |
| K₂HPO₄ | 0.7 |
| MgSO₄•7H₂O | 1.0 |
| MnSO₄•5H₂O | 0.01 |
| FeSO₄•7H₂O | 0.01 |
| Thiamine hydrochloride | 0.002 |
| Yeast extract | 2.0 |
| L-isolucine | 0.01 |
| CaCO₃ | 33.0 |

MgSO₄•7H₂O and CaCO₃ were each sterilized separately.

TABLE 1

| Strain | OD₅₄₀ | Thr, g/l |
|---|---|---|
| MG1655Δtdh, rhtA* (pVIC40)¹ | 30.5 ± 0.4 | 6.0 ± 0.3 |
| Km^{R} | | |
| MG1655Δtdh, rhtA*, P_{tac}nagE(pVIC40)² | 26.8 ± 0.7 | 7.3 ± 0.5 |
| Cm^{R}, Km^{R} | | |
| MG1655Δtdh, rhtA*, P_{tac}nagE, Δcrr (pVIC40)² | 26.5 ± 0.6 | 8.1 ± 0.3 |
| Cm^{R}, Km^{R}, Tet^{R} | | |

Notes:
¹data of 5 test-tubes fermentation,
²data of 12 test-tubes fermentation.

It can be seen from the Table 1, MG1655Δtdh, rhtA*, P_{tac}nagE accumulated a higher amount of L-threonine as compared with MG1655Δtdh, rhtA*, in which the expression amount of N-acetylglucosamine permease is not increased.

### Example 4

### Construction of E. coli MG1655Δtdh, rhtA*, P_{tac}nagE, Δcrr and Effect of Additional Inactivation of the crr Gene on L-Threonine Production

Strain MG1655Δtdh, rhtA*, P_{tac}nagE, Δcrr was constructed by inactivation of the native crr gene in E. coli MG1655Δtdh, rhtA*, P_{tac}nagE using the tetAtetR genes.

To substitute the native crr gene, a DNA fragment carrying tetracycline resistance marker (Tet^{R}) encoded by the tetAtetR genes was integrated into the chromosome of E. coli MG1655Δtdh, rhtA*, P_{tac}nagE by the method described by Datsenko K. A. and Wanner B. L. (Proc. Natl. Acad. Sci. USA, 2000, 97, 6640-6645).

A DNA fragment containing a tet^{R} marker encoded by the tetAtetR genes was obtained by PCR using the genomic DNA of E. coli Tn10 (VKPM B-5993) as the template, and primers P11 (SEQ ID NO: 13) and P12 (SEQ ID NO: 14). Each of primers contains 40 nucleotides homologous to the 5′-region and 3′-region of the crr gene accordingly which were introduced into the primers for further integration into the bacterial chromosome. The strain VKPM B-5993 is available from the Russian National Collection of Industrial Microorganisms (Russia, 113545 Moscow, 1 Dorozhny proezd, 1) upon request.

PCR was provided using the “Gene Amp PCR System 2700” amplificatory (Applied Biosystems). The reaction mixture (total volume—50 μl) consisted of 5 μl of 10×PCR-buffer with 25 mM MgCl₂ (“Fermentas”, Lithuania), 200 μM each of dNTP, 25 pmol each of the exploited primers and 1 U of Taq-polymerase (“Fermentas”, Lithuania). Approximately 20 ng of the genomic DNA was added to the reaction mixture as a template DNA for the PCR amplification. The temperature profile was the following: initial DNA denaturation for 5 min at 95° C., followed by 35 cycles of denaturation at 95° C. for 30 sec, annealing at 54° C. for 30 sec, elongation at 72° C. for 1 min 30 sec; and the final elongation for 5 min at 72° C. Then, the amplified DNA fragment was purified by agarose gel-electrophoresis, extracted using “GenElute Spin Columns” (“Sigma”, USA) and precipitated by ethanol.

The obtained DNA fragment was used for electroporation and Red-mediated integration into the bacterial chromosome of the E. coli MG1655/pKD46.

The conditions of the tetAtetR fragment electroporation into E. coli MG1655Δtdh,rhtA, P_{tac}nagE strain were the same as described above.

Shocked cells were added to 1 ml of SOC medium (Sambrook et al, “Molecular Cloning A Laboratory Manual, Second Edition”, Cold Spring Harbor Laboratory Press (1989)), incubated at 37° C. for 2 hours, and then spread onto L-agar containing 50 μg/ml of tetracycline.

Colonies grown within 24 hours were tested for the presence of Tet^{R} marker instead of the native crr gene by PCR using primers P13 (SEQ ID NO: 15) and P14 (SEQ ID NO: 16). For this purpose, a freshly isolated colony was suspended in 20 μl water and then 1 μl of obtained suspension was used for PCR. The temperature profile follows: initial DNA denaturation for 10 min at 95° C.; then 30 cycles of denaturation at 95° C. for 30 sec, annealing at 54° C. for 30 sec and elongation at 72° C. for 1.5 min; the final elongation for 5 min at 72° C. A few Tet^{R} colonies tested contained the desired 2040 bp DNA fragment, confirming the presence of Tet^{R} marker DNA instead of the native crr gene of 670 bp. One of the obtained strains was cured of the thermosensitive plasmid pKD46 by culturing at 37° C. and the resulting strain was named as E. coli MG1655Δtdh,rhtA,P_{tac}nagE, Δcrr.

E. coli MG1655Δtdh, rhtA*, P_{tac}nagE, Δcrr was cultivated at 37° C. for 18 hours in a nutrient broth and 0.3 ml of each of the obtained cultures was inoculated into 3 ml of fermentation medium in a 20×200 mm test tube and cultivated at 37° C. for 24 hours with a rotary shaker as described above. The results of test tube fermentation are shown in Table 1.

### Example 5

### Effect of Increasing the nagE Gene Expression on L-Lysine Production

To test the effect of enhanced expression of the nagE gene under the control of Plc promoter on L-lysine production, a DNA fragment comprising the nagE gene was cloned into pMW219 (Takara Shuzo, Japan). Specific procedures of constructing a plasmid used for enhancing expression of the nagE gene were as follows; DNA fragments comprising the nagE gene were amplified by PCR method, using primers P15 (SEQ ID NO: 17) and P16 (SEQ ID NO: 18), which included the HindIII and XbaI sites, respectively. PCR was provided using the “Pyrobest DNA Polymerase” (Applied Takara Shuzo, Japan). Approximately 20 ng of the genomic DNA of E. coli K-12 MG1655 was used as a template DNA for the PCR amplification. The temperature profile was the following: initial DNA denaturation for 1 min at 94° C., followed by 25 cycles of denaturation at 98° C. for 10 sec, annealing at 55° C. for 30 sec, elongation at 72° C. for 2 min; and the final elongation for 5 min at 72° C. Then, the amplified DNA fragment was purified by agarose gel-electrophoresis, extracted using “GenElute Spin Columns” (“Sigma”, USA) and precipitated by ethanol. The PCR product and the vector pMW219 were digested by HindIII and XbaI, and then ligated to each other by 2× Ligation Mit (Nippon Gene, Japan). Thus the plasmid pM-nagE containing the nagE gene under the control of the P_{lac} promoter was constructed.

The lysine-producing E. coli strain WC196 (pCABD2) was transformed with pMW219 for a control and pM-nagE, and WC196 (pCABD2, pMW219) and WC196 (pCABD2, pM-nagE) were constructed, respectively. pCABD2 is a plasmid comprising a dapA gene coding for a dihydrodipicolinate synthase having a mutation which desensitizes feedback inhibition by L-lysine, a lysC gene coding for aspartokinase III having a mutation which desensitizes feedback inhibition by L-lysine, a dapB gene coding for a dihydrodipicolinate reductase gene, and a ddh gene coding for diaminopimelate dehydrogenase (U.S. Pat. No. 6,040,160).

Both E. coli strains WC196 (pCABD2, pMW219) and WC196 (pCABD2, pM-nagE) were cultured in the L-medium containing 25 mg/l of kanamycin and 20 mg/l of streptomycin at 37° C. until OD₆₀₀ became about 0.6. Then, an equal volume of 40% glycerol solution was added to each culture broth, stirred, and then divided into appropriate volumes, and stored at −80° C. These are referred to herein as glycerol stocks.

The glycerol stocks of these strains were thawed, and 100 μl of each stock was uniformly plated on an L-plate containing 25 mg/l of kanamycin and 20 mg/l of streptomycin and incubated at 37° C. for 24 hours. About ⅛ of the cells collected from the plate were inoculated into 20 ml of the fermentation medium containing 25 mg/l of kanamycin and 20 mg/l of streptomycin in a 500 ml Sakaguchi flask, and cultured at 37° C. for 24 hours on a culturing apparatus with shaking by reciprocal movement. After cultivation, the amount of lysine which had accumulated in the medium was measured using Biotech Analyzer AS210 (Sakura Seiki). The results of the fermentation are shown in Table 2.

The composition of the fermentation medium (g/l) is as follows:

| | |
|---|---|
| Glucose | 40 |
| (NH₄)₂SO₄ | 24 |
| K₂HPO₄ | 1.0 |
| MgSO₄x7H₂O | 1.0 |
| FeSO₄x7H₂O | 0.01 |
| MnSO₄x5H₂O | 0.01 |
| Yeast extract | 2.0 |

pH was adjusted to 7.0 by KOH and the medium was autoclaved at 115° C. for 10 min. Glucose and MgSO₄×7H₂O were sterilized separately. 30 g/l of CaCO₃, which had been dry-heat sterilized at 180° C. for 2 hours, was added.

TABLE 2

| | | Lys•HCl, |
|---|---|---|
| Strain | OD₆₀₀ | g/l |
| WC196(pCABD2, pMW219) | 12.4 | 9.3 |
| WC196(pCABD2, pM-nagE) | 14.5 | 11.3 |

It can be seen from the Table 2, E. coli WC196 (pCABD2, pM-nagE) causes a higher amount of L-lysine accumulation as compared with E. coli WC196 (pCABD2, pMW219).

### Example 6

### Effect of Increasing the nagE Gene Expression on L-Histidine Production

To test the effect of enhanced expression of the nagE gene under the control of a P_{tac} promoter on hidtidine production, the DNA fragments from the chromosome of the above-described E. coli strain MG1655Δtdh, rhtA*, P_{tac}nagE can be transferred to histidine-producing E. coli strain 80 by P1 transduction (Miller, J. H. (1972) Experiments in Molecular Genetics, Cold Spring Harbor Lab. Press, Plainview, N.Y.). The strain 80 has been described in Russian patent 2119536 and deposited in the Russian National Collection of Industrial Microorganisms (Russia, 117545 Moscow, 1 Dorozhny proezd, 1) on Oct. 15, 1999 under the accession number VKPM B-7270 and then converted to a deposit under the Budapest Treaty on Jul. 12, 2004.

The resulting strain 80 P_{tac}nagE and parent strain 80 can each be cultivated in L broth for 6 hours at 29° C. Then 0.1 ml of obtained culture can be inoculated into 2 ml of fermentation medium in 20×200 mm test tube and cultivated for 65 hours at 29° C. with a rotary shaker (350 rpm). After cultivation, the amount of histidine which has accumulated in the medium can be determined by paper chromatography. The paper can be developed with a mobile phase: n-butanol:acetic acid:water=4:1:1 (v/v). A solution of ninhydrin (0.5%) in acetone can be used as a visualizing reagent.

The composition of the fermentation medium (pH 6.0) (g/l):

| | |
|---|---|
| Glucose | 100.0 |
| Mameno (soybean hydrolysate) | 0.2 as total nitrogen |
| L-proline | 1.0 |
| (NH₄)₂SO₄ | 25.0 |
| KH₂PO₄ | 2.0 |
| MgSO₄•7H₂0 | 1.0 |
| FeSO₄•7H₂0 | 0.01 |
| MnSO₄ | 0.01 |
| Thiamine | 0.001 |
| Betaine | 2.0 |
| CaCO₃ | 60.0 |

Glucose, proline, betaine and CaCO₃ are sterilized separately. pH is adjusted to 6.0 before sterilization.

### Example 7

### Effect of Increasing the nagE Gene Expression on L-Phenylalanine Production

To test the effect of enhanced expression of the nagE gene under the control of a P_{tac} promoter on phenylalanine production, the DNA fragments from the chromosome of the above-described E. coli strain MG1655Δtdh, rhtA*, P_{tac}nagE can be transferred to phenylalanine-producing E. coli strain AJ12739 by P1 transduction (Miller, J. H. (1972) Experiments in Molecular Genetics, Cold Spring Harbor Lab. Press, Plainview, N.Y.). The strain AJ12739 has been deposited in the Russian National Collection of Industrial Microorganisms (VKPM) (Russia, 113545 Moscow, 1 Dorozhny proezd, 1) on Nov. 6, 2001 under the accession number VKPM B-8197 and then converted to a deposit under the Budapest Treaty on Aug. 23, 2002.

The resulting strain AJ12739 P_{tac}nagE and parent strain AJ12739 can each be cultivated at 37° C. for 18 hours in a nutrient broth, and 0.3 ml of the obtained culture can be inoculated into 3 ml of a fermentation medium in a 20×200 mm test tube and cultivated at 37° C. for 48 hours with a rotary shaker. After cultivation, the amount of phenylalanine which has accumulated in the medium can be determined by TLC. 10×15 cm TLC plates coated with 0.11 mm layers of Sorbfil silica gel without fluorescent indicator (Stock Company Sorbpolymer, Krasnodar, Russia) can be used. Sorbfil plates can be developed with a mobile phase: propan-2-ol:ethylacetate:25% aqueous ammonia:water=40:40:7:16 (v/v). A solution (2%) of ninhydrin in acetone can be used as a visualizing reagent.

The composition of the fermentation medium (g/l):

| | |
|---|---|
| Glucose | 40.0 |
| (NH₄)₂SO₄ | 16.0 |
| K₂HPO₄ | 0.1 |
| MgSO₄•7H₂O | 1.0 |
| FeSO₄•7H₂O | 0.01 |
| MnSO₄•5H₂O | 0.01 |
| Thiamine HCl | 0.0002 |
| Yeast extract | 2.0 |
| Tyrosine | 0.125 |
| CaCO₃ | 20.0 |

Glucose and magnesium sulfate are sterilized separately. CaCO₃ dry-heat sterilized at 180° for 2 hours. pH is adjusted to 7.0.

### Example 8

### Substitution of the Native Promoter Region of the nagE Gene in E. Coli by Hybrid P_{L-tac} Promoter

To substitute the native promoter region of the nagE gene, a DNA fragment carrying a hybrid P_{L-tac} promoter and chloramphenicol resistance marker (Cm^{R}) encoded by the cat gene was integrated into the chromosome of the E. Coli MG1655 (ATCC 700926) in place of the native promoter region by the method described by Datsenko K. A. and Wanner B. L. (Proc. Natl. Acad. Sci. USA, 2000, 97, 6640-6645) which is also called as a “Red-mediated integration” or “Red-driven integration”. The recombinant plasmid pKD46 (Datsenko, K. A., Wanner, B. L., Proc. Natl. Acad. Sci. USA, 2000, 97, 6640-6645) having a thermosensitive replicon was used as the donor of the phage λ-derived genes responsible for the Red-mediated recombination system. E. Coli BW25113 containing the recombinant plasmid pKD46 can be obtained from the E. Coli Genetic Stock Center, Yale University, New Haven, USA, the accession number of which is CGSC7630.

The hybrid P_{L-tac} promoter was synthesized chemically. The nucleotide sequence of the substituted promoter is presented in the Sequence listing (SEQ ID NO: 19). The synthesized DNA fragment containing the hybrid P_{L-tac} promoter contains a BglII recognition site at the 5′-end thereof, which is necessary for further joining to the cat gene and 36 nucleotides homologous to the 5′-terminus of the nagE gene introduced for further integration into the bacterial chromosome.

A DNA fragment containing a Cm^{R} marker encoded by the cat gene was obtained by PCR using the commercially available plasmid pACYC184 (GenBank/EMBL accession number X06403, “Fermentas”, Lithuania) as the template, and primers P17 (SEQ ID NO: 20) and P18 (SEQ ID NO: 21). Primer P17 contains a BglII recognition site at the 5′-end thereof, which is necessary for further joining to the hybrid P_{L-tac} promoter, and primer P18 contains 36 nucleotides homologous to the region located 120 bp upstream of the start codon of the nagE gene, which were introduced into the primer for further integration into the bacterial chromosome.

PCR was provided using the “TermoHybaid PCR Express” amplificator. The reaction mixture (total volume—50 μl) consisted of 5 μl of 10×PCR-buffer with 15 mM MgCl₂ (“Fermentas”, Lithuania), 200 μM each of dNTP, 25 pmol each of the exploited primers and 1 U of Taq-polymerase (“Fermentas”, Lithuania). Approximately 5 ng of the plasmid DNA was added into the reaction mixture as a template DNA for the PCR amplification. The temperature profile was the following: initial DNA denaturation at 95° C. for 5 min, followed by 25 cycles of denaturation at 95° C. for 30 sec, annealing at 55° C. for 30 sec, elongation at 72° C. for 30 sec; and the final elongation at 72° C. for 7 min. Then, the amplified DNA fragment was purified by agarose gel-electrophoresis, extracted using “GenElute Spin Columns” (“Sigma”, USA), and precipitated by ethanol.

Each of the two above-described DNA fragments was treated with BglII restrictase and ligated. The ligation product was amplified by PCR using primers P18 (SEQ ID NO: 21) and P19 (SEQ ID NO: 22).

The amplified DNA fragment was purified by agarose gel-electrophoresis, extracted using “GenElute Spin Columns” (“Sigma”, USA), and precipitated by ethanol. The obtained DNA fragment was used for electroporation and Red-mediated integration into the chromosome of E. coli MG1655/pKD46.

MG1655/pKD46 cells were grown overnight at 30° C. in the liquid LB-medium containing ampicillin (100 μg/ml), then diluted 1:100 with the SOB-medium (Yeast extract, 5 g/l; NaCl, 0.5 g/l; Tryptone, 20 g/l; KCl, 2.5 mM; MgCl₂, 10 mM) containing ampicillin (100 μg/ml) and L-arabinose (10 mM) (arabinose is used for inducing the plasmid encoding genes of the Red system) and grown at 30° C. to reach the optical density of the culture OD₆₀₀=0.4-0.7. Grown cells from 10 ml of the culture were washed 3 times with the ice-cold de-ionized water, followed by suspending in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions. Shocked cells were added to 1 ml of SOC medium (Sambrook et al., “Molecular Cloning A Laboratory Manual, Second Edition”, Cold Spring Harbor Laboratory Press (1989)), incubated 2 hours at 37° C., and then were spread onto L-agar containing 25 μg/ml of chloramphenicol. Colonies grown within 24 hours were tested for the presence of Cm^{R} marker, instead of the native promoter region of the nagE gene by PCR using primers P20 (SEQ ID NO: 23) and P21 (SEQ ID NO: 24). For this purpose, a freshly isolated colony was suspended in 20 μl water and then 1 μl of the obtained suspension was used for PCR. The following temperature profile was used: initial DNA denaturation at 95° C. for 10 min; then 30 cycles of denaturation at 95° C. for 30 sec, annealing at 55° C. for 30 sec and elongation at 72° C. for 1 min; the final elongation at 72° C. for 7 min. A few Cm^{R} colonies tested contained the desired 1300 bp DNA fragment, confirming the presence of Cm^{R} marker DNA instead of 620 bp native promoter region of nagE gene. One of these strains was cured from the thermosensitive plasmid pKD46 by culturing at 37° C. and the resulting strain was named as E. coli MG1655 P_{L-tac}nagE.

### Example 9

### Effect of Increasing the nagE Gene Expression on L-Arginine Production

To test the effect of enhanced expression of the nagE gene under the control of a P_{L-tac} promoter on arginine production, the DNA fragments from the chromosome of the above-described E. coli strain MG1655 P_{L-tac}nagE was transferred to the arginine-producing E. coli strain 237 by P1 transduction (Miller, J. H. (1972), Experiments in Molecular Genetics, Cold Spring Harbor Lab. Press, Plainview, N.Y.). The strain 237 has been deposited in the Russian National Collection of Industrial Microorganisms (VKPM) (Russia, 113545 Moscow, 1 Dorozhny proezd, 1) on Apr. 10, 2000 under accession number VKPM B-7925 and then converted to a deposit under the Budapest Treaty on May 18, 2001.

The resulting strain 237 P_{L-tac}nagE and the parent strain 237 were each cultivated at 37° C. for 18 hours in 2 ml of LB nutrient broth, and 0.3 ml of the obtained culture was inoculated into 2 ml of fermentation medium in a 20×200 mm test tube, and cultivated at 34° C. for 72 hours on a rotary shaker.

After cultivation, the amount of L-arginine which has accumulated in the medium was determined by paper chromatography using the following mobile phase: butanol:acetic acid:water=4:1:1 (v/v). A solution (2%) of ninhydrin in acetone was used as a visualizing reagent. A spot containing L-arginine can be cut off, L-arginine was eluted in 0.5% water solution of CdCl₂, and the amount of L-arginine was estimated spectrophotometrically at 540 nm. The results of the fermentation are shown in Table 3.

The composition of the fermentation medium (g/l):

| | |
|---|---|
| Glucose | 50.0 |
| (NH4)₂SO₄ | 25.0 |
| KH₂PO₄ | 2.0 |
| MgSO₄•7H₂O | 1.0 |
| Thiamine HCl | 0.0001 |
| Yeast extract | 5.0 |
| L-isoleucine | 0.05 |
| CaCO₃ | 15.0 |

Glucose and magnesium sulfate were sterilized separately. CaCO₃ was dry-heat sterilized at 180° C. for 2 hours. pH was adjusted to 7.0.

TABLE 3

| Strain | OD₅₄₀ | Arg, g/l |
|---|---|---|
| 237 | 20.4 ± 0.4 | 3.5 ± 0.2 |
| 237::P_{L-tac}nagE | 20.2 ± 0.5 | 5.2 ± 0.3 |

It can be seen from the Table 3, E. coli 237 P_{L-tac}nagE accumulated a higher amount of L-arginine as compared with E. coli 237.

### Example 10

### Effect of Increasing the nagE Gene Expression on L-Tryptophan Production

To test the effect of enhanced expression of the nagE gene under the control of a P_{L-tac} promoter on tryptophan production, the DNA fragments from the chromosome of the above-described E. coli strain MG1655P_{L-tac}nagE was transferred to tryptophan-producing E. coli SV164 (pGH5) by P1 transduction (Miller, J. H. (1972) Experiments in Molecular Genetics, Cold Spring Harbor Lab. Press, Plainview, N.Y.). The strain SV164 (pGH5) is described in detail in U.S. Pat. No. 6,180,373.

The resulting strain SV164(pGH5) P_{L-tac}nagE and the parent strain SV164(pGH5) were each cultivated with shaking at 37° C. for 18 hours in a 3 ml of nutrient broth supplemented with 20 mg/ml of tetracycline (marker of pGH5 plasmid). 0.3 ml of the obtained cultures was inoculated into 3 ml of a fermentation medium containing tetracycline (20 mg/ml) in 20×200 mm test tubes, and cultivated at 37° C. for 48 hours with a rotary shaker at 250 rpm. After cultivation, the amount of tryptophan which has accumulated in the medium was determined by TLC as described in Example 7. The composition of the fermentation medium is presented in Table 4. The results of the fermentation are shown in Table 5.

TABLE 4

| Groups | Component | Final concentration, g/l |
|---|---|---|
| A | KH₂PO₄ | 1.5 |
| | NaCl | 0.5 |
| | (NH₄)₂SO₄ | 1.5 |
| | L-Methionine | 0.05 |
| | L-Phenylalanine | 0.1 |
| | L-Tyrosine | 0.1 |
| | Mameno (total N) | 0.07 |
| B | Glucose | 40.0 |
| | MgSO₄•7H₂O | 0.3 |
| C | CaCl₂ | 0.011 |
| D | FeSO₄•7H₂O | 0.075 |
| | Sodium citrate | 1.0 |
| E | Na₂MoO₄•2H₂O | 0.00015 |
| | H₃BO₃ | 0.0025 |
| | CoCl₂•6H₂O | 0.00007 |
| | CuSO₄•5H₂O | 0.00025 |
| | MnCl₂•4H₂O | 0.0016 |
| | ZnSO₄•H₂O | 0.0003 |
| F | Thiamine HCl | 0.005 |
| G | CaCO₃ | 30.0 |
| H | Pyridoxine | 0.03 |

Group A had pH of 7.1, adjusted by NH₄OH. Each group was sterilized separately.

TABLE 5

| Strain | OD₅₄₀ | Trp, g/l |
|---|---|---|
| SV164 (pGH5) | 13.3 ± 0.7 | 4.6 ± 0.1 |
| SV164::P_{L-tac}nagE (pGH5) | 16.2 ± 0.4 | 5.2 ± 0.1 |

It can be seen from the Table 5, E. coli SV164 P_{L-tac}nagE (pGH5) accumulated a higher amount of L-tryptophan as compared with E. coli SV164 (pGH5).

### Example 11

### Effect of Increasing the nagE Gene Expression on L-Glutamic Acid Production

To test the effect of enhanced expression of the nagE gene under the control of a P_{tac} promoter on glutamic acid production, the DNA fragments from the chromosome of the above-described E. coli strain MG1655Δtdh, rhtA*, P_{tac}nagE can be transferred to glutamic acid-producing E. coli strain VL334thrC⁺ by P1 transduction (Miller, J. H. (1972) Experiments in Molecular Genetics, Cold Spring Harbor Lab. Press, Plainview, N.Y.). The strain VL334thrC⁺ is described in detail in EP1172433.

The resulting strain VL334thrC⁺ P_{tac}nagE and parent strain VL334thrC⁺ can each be cultivated with shaking at 37° C. for 18 hours in a 3 ml of nutrient broth. 0.3 ml of the obtained cultures can be inoculated into 3 ml of a fermentation medium in 20×200 mm test tubes, and cultivated at 37° C. for 48 hours with a rotary shaker at 250 rpm.

The composition of the fermentation medium (pH 7.2) (g/l):

| | |
|---|---|
| Glucose | 60.0 |
| Ammonium sulfate | 25.0 |
| KH₂PO₄ | 2.0 |
| MgSO₄ | 1.0 |
| Thiamine | 0.0001 |
| L-isoleucine | 0.05 |
| CaCO₃ | 25.0 |

Glucose and CaCO₃ can be sterilized separately.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

### INDUSTRIAL APPLICABILITY

According to the present invention, production of L-amino acids, such as L-threonine, L-lysine, L-histidine, L-phenylalanine, L-arginine, L-tryptophan, and L-glutamic acid of a bacterium of the Enterobacteriaceae family can be enhanced.

We claim:

1. An L-amino acid-producing bacterium of the Enterobacteriaceae family, wherein said bacterium has been modified to enhance an activity of N-acetylglucosamine permease.

2. The bacterium according to claim 1, wherein the activity of N-acetylglucosamine permease is enhanced by increasing the expression of a gene which encodes N-acetylglucosamine permease.

3. The bacterium according to claim 2, wherein said activity of N-acetylglucosamine permease is enhanced by modifying an expression control sequence of the gene encoding N-acetylglucosamine permease or by increasing the copy number of the gene encoding N-acetylglucosamine permease.

4. The bacterium according to claim 1, wherein said bacterium has been additionally modified to enhance an activity of glucokinase.

5. The bacterium according to claim 1, where said bacterium has been additionally modified to enhance an activity of xylose isomerase.

6. The bacterium according to claim 1, wherein the bacterium is selected from the group consisting of the genera Escherichia, Enterobacter, Erwinia, Klebsiella, Pantoea, Providencia, Salmonella, Serratia, Shigella, and Morganella.

7. The bacterium according to claim 1, wherein said gene encodes an N-acetylglucosamine permease selected from the group consisting of:
(A) a protein which comprises the amino acid sequence of SEQ ID NO: 2, and
(B) a variant protein of the amino acid sequence shown in SEQ ID NO: 2, and which has an activity of N-acetylglucosamine permease.

8. The bacterium according to claim 2, wherein said gene encoding N-acetylglucosamine permease comprises a DNA selected from the group consisting of:
(a) a DNA which comprises a nucleotide sequence of nucleotides 1 to 1947 in SEQ ID NO: 1, and
(b) a DNA which is hybridizable with a nucleotide sequence of nucleotides 1-1947 in SEQ ID NO: 1, or a probe which can be prepared from said nucleotide sequence under stringent conditions, and encodes a protein having an activity of N-acetylglucosamine permease.

9. The bacterium according to claim 8, wherein said stringent conditions comprise those in which washing is performed at 60° C. at a salt concentration of 1×SSC and 0.1% SDS, for approximately 15 minutes.

10. The bacterium according to claim 1, wherein said bacterium is an L-threonine producing bacterium.

11. The bacterium according to claim 10, wherein said bacterium has been additionally modified to enhance expression of a gene selected from the group consisting of
the mutant thrA gene which codes for aspartokinase homoserine dehydrogenase I and is resistant to feedback inhibition by threonine,
the thrB gene which codes for homoserine kinase,
the thrC gene which codes for threonine synthase,
the rhtA gene which codes for a putative transmembrane protein,
the asd gene which codes for aspartate-β-semialdehyde dehydrogenase,
the aspC gene which codes for aspartate aminotransferase (aspartate transaminase), and combinations thereof.

12. The bacterium according to claim 11, wherein said bacterium has been modified to increase expression of said mutant thrA gene, said thrB gene, said thrC gene, and said rhtA gene.

13. The bacterium according to claim 11, wherein said bacterium has been additionally modified so that the crr gene which codes for catabolite repression regulator is inactivated.

14. The bacterium according to claim 13, wherein said bacterium has been modified to increase expression of said mutant thrA gene, said thrB gene, said thrC gene, and said rhtA gene.

15. The bacterium according to claim 1, wherein said bacterium is an L-lysine producing bacterium.

16. The bacterium according to claim 1, wherein said bacterium is an L-histidine producing bacterium.

17. The bacterium according to claim 1, wherein said bacterium is an L-phenylalanine producing bacterium.

18. The bacterium according to claim 1, wherein said bacterium is an L-arginine producing bacterium.

19. The bacterium according to claim 1, wherein said bacterium is an L-tryptophan producing bacterium.

20. The bacterium according to claim 1, wherein said bacterium is an L-glutamic acid producing bacterium.

21. A method for producing an L-amino acid which comprises cultivating the bacterium according to claim 1 in a culture medium to cause accumulation of the L-amino acid in the culture medium, and isolating the L-amino acid from the culture medium.

22. The method according to claim 21, wherein said L-amino acid is selected from the group consisting of L-threonine, L-lysine, L-histidine, L-phenylalanine, L-arginine, L-tryptophan, and L-glutamic acid.
